(19) United States
(12) Patent Application Publication
(10) Pub. No.: US 2008/0119846 A1
(43) Pub. Date: May 22, 2008

(54) METHODS AND APPARATUS FOR PERCUTANEOUS PATIENT ACCESS AND SUBCUTANEOUS TISSUE TUNNELING

(76) Inventor: Robert F. Rioux, Ashland, MA (US)

Correspondence Address: Vista IP Law Group LLP, 2040 MAIN STREET, 9TH FLOOR, IRVINE, CA, 92614, US

(21) Appl. No.: 11/871,089

(22) Filed: Oct. 11, 2007

Related U.S. Application Data
(60) Provisional application No. 60/829,129, filed on Oct. 11, 2006.

(51) Int. Cl. A61B 18/08

(52) U.S. Cl. 606/41

(57) ABSTRACT

Methods and apparatus for percutaneous blood vessel access and tissue tunneling, including guidewire placement, are provided. In one embodiment, an atraumatic distal tip of a tissue tunneling, medical probe is pressed against the derma, and electrical energy (e.g., radio frequency (RF) energy) is conveyed to or from the distal tip to ablate tissue immediately adjacent the distal tip, thereby advancing the probe through the derma while the tissue immediately adjacent the distal tip is ablated. In one embodiment, the medical probe comprises an elongated, rigid, electrically conductive, shaft, and an electrically insulative sheath disposed on the shaft to form an exposed tip electrode configured for electrosurgically ablating solid tissue located immediately axial to the tip electrode to facilitate rapid advancement of the medical probe through solid tissue. The probe may include an axially extending lumen through which an elongated guidewire may be introduced.

### RELATED APPLICATION DATA

This application claims the benefit under 35 U.S.C. § 119 to provisional application Ser. No. 60/829,129, filed Oct. 11, 2006.

### FIELD OF THE INVENTION

The field of the invention relates to tissue penetrating probes, and in particular, to percutaneous and subcutaneous probes.

### BACKGROUND

There are many instances where inadvertent injury to a healthcare worker (HCW) or to a patient may occur when using or handling conventional needles or tissue dissection devices.

For example, despite the many precautions and guidelines issued by the Department of Health and professional organizations, and well as the many commercially available safety devices that have been developed to prevent needle sticks and sharps injuries, HCWs suffer between 600,000 and 1,000,000 injuries per year caused by the mishandling of conventional needles and sharps. Significantly, exposure to pathogens carried by such needles may lead to serious infections, such as hepatitis B, hepatitis C, and Human Immunodeficiency Virus (HIV)—the virus that causes Acquired Immunodeficiency syndrome (AIDS). At least 1,000 HCWs are estimated to contract one of these serious infections annually from needle sticks and sharps injuries per year.

As another example, tissue dissection devices, such as tunnelers, necessarily cause inadvertent damage when being advanced between the tissue layers of a patient. Current tissue tunnelers are blunt tips rods that a physician uses to forcefully create a tunnel through tissue or between tissue layers, thereby allowing another medical conduit, such as a catheter or electrical lead, to be subcutaneously routed through the patient's body. The process often requires moderate force in order to tear through soft tissue. Oftentimes, the tunneling member gets caught on fibrous tissue that is difficult to push through and requires a knife or scissors to cut. The pushing and tearing of the tissues is the most painful aspect of the procedure. Besides causing pain, the tearing of tissue may also cause other complications, such as bleeding and the inadvertent entrance of the tunneling member into other tissue structures of the patient's body.

For these reasons, it would be desirable to provide atraumatic medical devices that can be introduced through tissue without inadvertently causing significant damage to the tissue.

### SUMMARY OF THE INVENTION

In accordance with a first aspect of the present inventions, a method of percutaneously accessing a patient with an elongated probe, is provided. The probe is preferably one that is conducive to minimally invasive procedures, e.g., one having a size within the range of 27 gauge and 8 Fr, preferably within the range of 24 gauge to 15 gauge. The method comprises placing an atraumatic distal tip of the probe against the derma of the patient, conveying electrical energy to or from the distal tip to ablate tissue immediately adjacent the distal tip, and advancing the probe through the derma while the tissue immediately adjacent the distal tip is ablated. Thus, it can be appreciated that the atraumatic distal tip of the probe prevents or minimizes accidental needle sticks, while allowing percutaneous access to a patient on-demand.

In one method, the electrical energy takes the form of electromagnetic energy, such as radio frequency (RF) energy. The power level of the electrical energy may be in the range of 1 W to 50 W, but more often will be in the range of 5 W to 30 W. The probe may include an electrically conductive shaft and an electrically insulative coating disposed on the shaft, so that the electrical energy is only conveyed to or from the distal tip. The distal tip to which or from which the electrical energy is conveyed may be relatively small to provide only the tissue ablation necessary to allow percutaneous advancement of the probe. In this case, the power level of the electrical energy may be relatively low, e.g., equal to or less than 30 W, and in most cases, equal to or less than 10 W. In one method, the tissue immediately axial to the distal tip is ablated. Any ablation of tissue immediately radial to the distal tip may be eliminated or minimized to the nature of the distal tip. For example, in one method, any tissue ablated immediately radial to the distal tip is limited to a depth of 1 mm, and preferably 0.1 mm, from the surface of the distal tip.

The probe may be percutaneously advanced into the patient to perform any one of a variety of medical procedures. For example, the probe can be used as part of an introducer system for a catheter, in which case, a guidewire can be introduced through the probe and/or a cannula introduced over the probe. The probe can include a cannula and inner member that can be removed as part of the coaxial introducer system for another probe, such as a biopsy probe. The probe can even be used as a guidewire itself. In another method, the electrical energy may be conveyed between the distal tip of the guidewire and a catheter having the guidewire disposed therethrough, in which case, the method may further comprise advancing the catheter with the guidewire through the derma while the tissue immediately adjacent the distal tip is ablated.

In another application, the probe may be used to introduce a liquid, such as drugs, into the patient, or may be used to remove liquid, such as blood, from the patient. The distal tip of the probe may be percutaneously located anywhere within the patient's body, either intravascularly or extravascularly. Optionally, the method may further comprise sensing physiological information adjacent the distal tip of the probe, and determining the nature of tissue in which the distal tip is located based on the sensed physiological information. By way of non-limiting example, such a feature provides an indication of whether the distal tip is located in a target blood vessel.

In accordance with a second aspect of the present inventions, a method of subcutaneously creating a tunnel through tissue with an elongated probe is provided. The method comprises conveying electrical energy to or from an atraumatic distal tip of the tunneling probe to ablate tissue immediately adjacent the distal tip, and subcutaneously advancing the tunneling probe within the patient while the tissue immediately adjacent the distal tip is ablated. In this manner, brute force need not be axially applied to the tunneling probe to traverse fibrous tissue, thereby minimizing pain and other complications associated with ripping or cutting through tissue. The natural coagulation effect of the electromagnetic energy may also prevent blood loss within the resulting tunnel.

In one method, the electromagnetic energy takes the form of radio frequency (RF) energy in the range of 30 W-100 W. The tunneling probe may include an electrically conductive shaft and an electrically insulative coating disposed on the shaft, so that the electrical energy is only conveyed to or from the distal tip. Optionally, the method may further comprise sensing physiological information adjacent the distal tip of the tunneling probe, and determining the nature of tissue in which the distal tip is located based on the sensed physiological information. By way of non-limiting example, such a feature provides an indication of whether the distal tip has veered out of the intended subcutaneous path into an unintended tissue structure, such as a chest wall.

In accordance with a third aspect of the present inventions, a medical probe is provided. The medical probe comprises an elongated, rigid, electrically conductive, shaft, and an electrically insulative sheath disposed on the shaft to form an exposed atraumatic tip electrode configured for electrosurgically ablating solid tissue located immediately axial to the tip electrode to facilitate rapid advancement of the medical probe through the solid tissue without substantially ablating solid tissue immediately radial to the tip electrode. The medical probe is preferably one that is conducive to minimally invasive procedures, e.g., one having a size within the range of 27 gauge and 8 Fr, preferably within the range of 24 gauge to 15 gauge.

In one embodiment, the exposed tip electrode is confined to a distal-facing surface of the shaft to ensure that tissue ablation is only performed in the distal direction. In another embodiment, the tip electrode is configured for ablating the solid tissue distal to the tip electrode when electrical energy at a power level of 30 W or less is applied to a proximal end of the shaft. In some embodiments, a power level of 10 W may be sufficient to ablate the solid tissue distal to the tip electrode. The medical probe may include an electrical connector to provide a convenient means for delivering electrical energy to the shaft of the medical probe. The medical probe may include a lumen extending through the shaft for, e.g., delivering medical implements, such as guidewire or drugs, or withdrawing fluids from the patient.

The medical probe may optionally be included within a percutaneous access kit, e.g., to provide a means for introducing a catheter within the patient. In this case, the percutaneous kit may include a guidewire configured for being introduced through the medical probe and/or an introducer cannula configured for being introduced over the medical probe. The probe can include a cannula and inner member that can be removed as part of the coaxial introducer system for another probe, such as a biopsy probe. The probe can even be used as a guidewire itself. The medical probe may also be included in a system comprising a source of electrical energy electrically coupled to the medical probe. The medical probe may optionally comprise a sensor carried by the shaft adjacent the tip electrode, wherein the sensor is configured for sensing a physiological parameter indicative of a tissue characteristic.

In accordance with a fourth aspect of the present inventions, another medical probe is provided. The medical probe comprises an elongated, rigid, electrically conductive, shaft, and an electrically insulative sheath disposed on the shaft to form an exposed atraumatic tip electrode configured for ablating solid tissue located immediately axial to the tip electrode to facilitate rapid advancement of the medical probe through the solid tissue when electrical energy at a level of 30 W or less is applied to a proximal end of the shaft. The detailed structure and function of the medical probe, and the use thereof in percutaneous access kits and systems, can be similar to the previously described medical probe.

In accordance with a fifth aspect of the present inventions, still another medical probe is provided. The medical probe comprises an elongated, electrically conductive, shaft, a lumen extending through the shaft, and an electrically insulative sheath disposed on the shaft to form an exposed atraumatic tip electrode configured for electrosurgically ablating solid tissue located immediately axial to the tip electrode to facilitate rapid advancement of the medical probe through the solid tissue. The detailed structure and function of the medical probe, and the use thereof in percutaneous access kits and systems, can be similar to the previously described medical probe.

In accordance with a sixth aspect of the present inventions, a method of percutaneously introducing a catheter having a guidewire disposed therethrough into a patient is provided. The method comprises placing an atraumatic distal tip of the guidewire against the derma of the patient, conveying electrical energy to or from the distal tip to ablate tissue immediately adjacent the distal tip, and advancing the catheter with the guidewire through the derma while the tissue immediately adjacent the distal tip is ablated. The characteristics of the electrical energy conveyed and the resulting tissue ablation can be similar as those described above. In an optional method, the electrical energy is conveyed between the distal tip of the guidewire and the catheter (e.g., a distal tip of the catheter).

In one method, the catheter with the guidewire is advanced into a blood vessel of the patient. Physiological information may optionally be sensed adjacent the distal of the guidewire, and the nature of the tissue in which the guidewire is located determined based on the sensed physiological information. The catheter and the guidewire may be advanced through the derma as a single integrated device. Once introduced within the patient, the method may further comprises axially moving the guidewire within the patient relative to the catheter, and then axially advancing the catheter along the guidewire within the patient.

In accordance with a seventh aspect of the present inventions, a catheter assembly is provided. The catheter assembly comprises a flexible therapeutic or diagnostic catheter having an elongated shaft and a lumen axially extending through the catheter shaft, and a flexible guidewire configured for being removably introduced through the catheter lumen. The guidewire has an elongated, electrically conductive, shaft and an electrically insulative sheath disposed on the shaft to form an exposed atraumatic tip electrode that extends from a distal end of the catheter shaft when the guidewire is inserted within the catheter lumen. The tip electrode is configured for electrosurgically ablating solid tissue located immediately axial to the tip electrode to facilitate rapid advancement of the catheter through solid tissue.

In an optional embodiment, the catheter includes an electrode disposed on the distal end of the catheter shaft, in which case, the tip electrode of the guidewire and catheter electrode can form a bipolar arrangement. In another embodiment, the catheter assembly comprises an electrical connector electrically coupled to the guidewire shaft. The detailed structure and function of the tip electrode can be the same as the previously described tip electrodes. In this case, the catheter assembly may also comprise a handle carrying the electrical connector and removably mounted to the guidewire shaft.

The catheter assembly may optionally comprise a locking mechanism configured for alternately affixing the guidewire relative to the catheter and allowing the guidewire to move relative to the catheter. The catheter assembly may also comprise a sensor carried by the guidewire shaft adjacent the tip electrode for sensing a physiological parameter indicative of a tissue characteristic. The catheter assembly may be included in a system comprising a source of electrical energy electrically coupled to the guidewire.

In accordance with an eighth aspect of the present inventions, a method of obtaining a sample from a tissue region using a biopsy probe having a tissue cutting element and a tissue containment element, is provided. The method comprises placing the biopsy probe adjacent the tissue region, such that a portion of a tissue volume prolapses into the containment element. The method further comprises conveying electrical energy (e.g., RF energy) to or from the cutting element while moving the cutting element relative to the tissue containment element (e.g., moving the cutting element distally relative to the containment element and/or over the containment element) to cut the prolapsed tissue portion away from a remainder of the tissue volume.

In one method, the electrical energy is conveyed only to or from a distal edge of the cutting element. In an optional method, the cut tissue is retained within the cutting element. Another optional method further comprises conveying electrical energy to or from the containment element to ablate tissue immediately distal to the biopsy probe, and advancing the biopsy probe through tissue to the tissue region while the tissue immediately distal to the biopsy probe is ablated.

In accordance with a ninth aspect of the present inventions, a biopsy probe is provided. The biopsy probe comprises an elongated cannula, and an elongated inner shaft disposed within the cannula. The biopsy probe further comprises a tissue containment element disposed on one of the cannula and inner shaft, and configured for allowing a portion of a volume of tissue to prolapse therein. The biopsy probe further comprises a cutting electrode (e.g., a cylindrical cutting electrode) disposed on another of the cannula and inner shaft, wherein the cutting electrode is configured for electrosurgically cutting the prolapsed tissue portion away from a remainder of the tissue volume when the cannula and inner shaft are moved relative to each other.

In one embodiment, the cannula or inner shaft includes an electrically conductive shaft and an electrically insulative sheath disposed over the conductive shaft to form the cutting electrode at the distal end thereof. In another embodiment, the cutting electrode is configured for electrosurgically cutting the prolapsed tissue portion away from the remainder of the tissue volume when the cannula is distally moved relative to the inner shaft and/or over the inner shaft. In this case, the cutting electrode may have a distal cutting edge. The biopsy probe may comprise an electrical connector electrically coupled to the cutting electrode.

In an optional embodiment, the biopsy probe further comprises an atraumatic tip electrode disposed on the other of the cannula and inner shaft, wherein the tip electrode is configured for electrosurgically ablating solid tissue located immediately axial to the tip electrode to facilitate rapid advancement of the biopsy probe through the solid tissue. In this case, the cannula or inner shaft may include an electrically conductive shaft and an electrically insulative sheath disposed over the conductive shaft to form the tip electrode at the distal end thereof. In another optional embodiment, the cannula or inner shaft is configured for retaining the cut tissue portion. The biopsy probe may be included in a system comprising a source of electrical energy electrically coupled to the biopsy probe.

Other and further aspects and features of the invention will be evident from reading the following detailed description of the preferred embodiments, which are intended to illustrate and not limit the present inventions.

### BRIEF DESCRIPTION OF DRAWINGS

The drawings illustrate the design and utility of embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a perspective view of a percutaneous catheter introduction system constructed in accordance with one embodiment of the present inventions;

FIG. 2 is a partially cutaway perspective view of the distal end of a percutaneous medical probe used in the system of FIG. 1;

FIG. 3 is a cross-sectional view of the percutaneous medical probe of FIG. 2, taken along line 3-3;

FIG. 4 is a partially cutaway perspective view of the distal end of another percutaneous medical probe used in the system of FIG. 1;

FIG. 5 is a partially cutaway perspective view of the distal end of still another percutaneous medical probe used in the system of FIG. 1;

FIG. 6 is a partially cutaway perspective view of the distal end of yet another percutaneous medical probe used in the system of FIG. 1;

FIG. 7 is a perspective view of an alternative percutaneous medical probe that can be used in the system of FIG. 1;

FIG. 8 is a cross-sectional view of the percutaneous medical probe of FIG. 7, taken along line 8-8;

FIGS. 9A-9H are side views illustrating a method of extravascularly introducing a catheter within a patient to drain a tissue region using the system of FIG. 1;

FIGS. 10A-10E are side views illustrating a method of intravascularly introducing a catheter within a patient using the system of FIG. 1;

FIG. 11 is a plan view of a catheter assembly constructed in accordance with the present inventions, wherein the catheter assembly is particularly shown disassembled;

FIG. 12 is a plan view of the catheter assembly of FIG. 11, wherein the catheter assembly is particularly shown assembled;

FIG. 13 is a cross-sectional view of the catheter assembly of FIG. 12, taken along the line 13-13;

FIG. 14 is a plan view of another catheter assembly constructed in accordance with the present inventions, wherein the catheter assembly is particularly shown disassembled;

FIG. 15 is a plan view of the catheter assembly of FIG. 14, wherein the catheter assembly is particularly shown assembled;

FIG. 16 is a cross-sectional view of the catheter assembly of FIG. 12, taken along the line 13-13;

FIG. 17 is a close-up view of the distal end of the catheter assembly of FIG. 12;

FIGS. 18A-18E are side views illustrating a method of intravascularly introducing the catheter assembly of FIG. 11, or alternatively the catheter assembly of FIG. 14, within a patient;

FIG. 19 is a partially cutaway, perspective view, of a biopsy probe constructed in accordance with one embodiment of the present inventions;

FIG. 20 is a cross-sectional view of the biopsy probe of FIG. 19, taken along the line 20-20;

FIG. 21 is a close-up perspective view of the distal end of the biopsy probe of FIG. 19, particularly showing one embodiment of a tissue containment element;

FIG. 22 is a close-up perspective view of a distal end of the biopsy probe of FIG. 19, particularly showing another embodiment of a tissue containment element;

FIGS. 23A-23F are side views illustrating a method of removing a tissue sample from a patient using the biopsy probe of FIG. 19 and medical probe of FIG. 7;

FIG. 24 is a plan view of a tissue tunneling system constructed in accordance with one embodiment of the present inventions;

FIG. 25 is a cross-sectional view of a subcutaneous tunneling medical probe used in the system of FIG. 7, taken along line 21-21; and

FIGS. 26A-26D are side views illustrating a method of creating a tunnel through tissue of a patient using the system of FIG. 24.

### DETAILED DESCRIPTION OF THE ILLUSTRATED EMBODIMENTS

Referring to FIG. 1, a percutaneous catheter introduction system 10 constructed in accordance with one embodiment of the present inventions will now be described. The catheter introducer system 10 generally comprises a catheter introducer kit 12 and a tissue ablation source, and in particular a radio frequency (RF) generator 14, configured for supplying RF energy in a controlled manner to the tissue ablative component of the introducer kit 12 via a RF cable 16.

The introducer kit 12 generally comprises (1) an RF-activated percutaneous probe 18 that can be percutaneously introduced through the derma of a patient; (2) a safety guidewire 20 that can be advanced through the percutaneous probe 18; (3) an introducer cannula sheath 22 that can be advanced over the safety guidewire 20; (4) a dilator/catheter assembly 24 for facilitating the percutaneous advancement of the introducer cannula sheath 22 over the safety guidewire 20 and into the patient; and (5) a working guidewire 26 that can be introduced through the introducer cannula sheath 22 and over which a flexible catheter (not shown) can be introduced.

In many respects, the components of the catheter introducer kit 12, with the exception of the percutaneous probe 18, are conventional. For example, the general structure of the components in the catheter introducer kit 12 may resemble those contained in the AccuStick™ II Introducer System, commercially available from Boston Scientific Corporation. Instead of relying on a sharpened traumatic tip, however, the percutaneous probe 18 relies on electrical energy, and in particular radio frequency (RF) energy, to facilitate percutaneous introduction into a patient. In this manner, the distal tip of the percutaneous probe 18 may be atraumatic in order to prevent or minimize the risk of inadvertent needle sticks. For the purposes of this specification, an element is atraumatic if it, when not energized with electrical current, does not pierce solid tissue when an amount of axial force otherwise sufficient to pierce solid tissue with a conventional needle, is applied to the element.

The percutaneous probe 18 may be a relatively small diameter needle designed to minimize tissue trauma during the percutaneous introduction thereof. To this end, the size of the percutaneous probe 18 may fall within the size range from 27 gauge up to 8 F, but preferably falls within the size range of 25 gauge to 15 gauge. In the illustrated embodiment, the size of the percutaneous probe 18 is 21 gauge. The length of the percutaneous probe 18 is consistent with typical needles used in introducer cannula assemblies, typically in the range from 5 cm to 30 cm, preferably from 10 cm to 25 cm. The structure of the percutaneous probe 18 will be described in further detail below.

The safety guidewire 20 is a relatively small diameter guidewire, e.g., having a diameter of 0.018″, configured to be introduced through a lumen of the small diameter percutaneous probe 18. The safety guidewire 20 may be supplied as an extra stiff guidewire or an optional nitinol guidewire designed to provide strength during tough procedures. The working guidewire 26 is a heavy-duty guidewire, e.g., having a diameter of 0.038″, configured to be introduced through a lumen of the introducer cannula sheath 22. The working guidewire 26 may, e.g., have a straight tip or J-shaped tip (as illustrated in FIG. 1).

Once the percutaneous probe 18 is removed from the safety guidewire 20, the introducer cannula sheath 22 and dilator/catheter assembly 24 are designed to be coaxially introduced over the safety guidewire 20. The introducer cannula sheath 22 has a suitable size, e.g., 6 F, that facilitates the placement of the working guidewire 26 alongside the safety guidewire 20. The distal end of the introducer cannula sheath 22 may carry a radiopaque marker (not shown) to enhance its visualization for more precise placement. The dilator/catheter assembly 24 has a dilator 28 having a tapered tip designed to facilitate introduction of the introducer cannula sheath 22, and a stiffening cannula 30 designed to provide the introducer cannula sheath 22 with the necessary axial strength during its introduction into the patient. The dilator/catheter assembly 24 may have a size, e.g., 4 F, suitable for introduction within the introducer cannula sheath 22.

The introducer cannula sheath 22 and dilator/catheter assembly 24 may be mated together in a locking arrangement to facilitate the percutaneous introduction thereof as a single integrated device. To this end, proximal adapters 32, 34, and 36 are provided at the proximal ends of the introducer cannula sheath 22, cannula 30, and dilator 28. In the illustrated embodiment, the sheath proximal adapter 32 and cannula proximal adapter 34 have respective male and female portions that removably mate with each other, e.g., in a threaded arrangement. Similarly, the cannula proximal adapter 34 and dilator proximal adapter 36 have respective male and female portions that removably mate with each other, e.g., in a threaded arrangement.

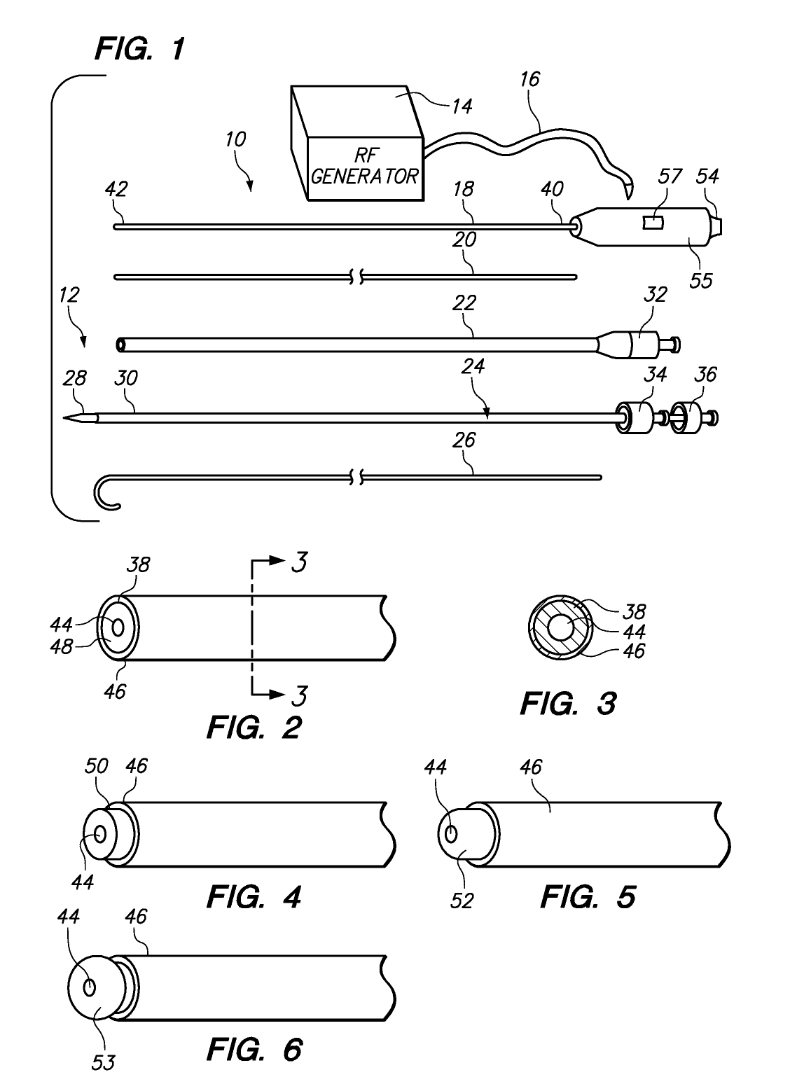

Referring further to FIGS. 2 and 3, the percutaneous probe 18 will be described in further detail. The percutaneous probe 18 comprises an elongated shaft 38 having a proximal end 40 and a distal end 42. In the embodiment illustrated in FIG. 2, the shaft 38 is rigid. For the purposes of this specification, a shaft of a probe is rigid if it is generally not suitable to be advanced along a tortuous anatomical conduit of a patient, as contrasted to, e.g., guidewires and intravascular catheters.

The shaft 38 is also composed of an electrically conductive material suitable to conduct electrical energy, such as RF energy, from the proximal end 40 to the distal end 42 thereof to effect an ablation function, as will be described in further detail below. Stainless steel is a suitable rigid and electrically conductive material. As illustrated in FIG. 2, the distal end 42 of the shaft 38 is atraumatic, so that the percutaneous probe 18 will not pierce the derma of the patient or healthcare worker when not energized with RF energy. The percutaneous probe 18 includes a guidewire lumen 44 extending through the axial center of the shaft 38.

The percutaneous probe 18 further comprises a sheath 46 disposed on the shaft 38. The sheath 46 is composed of a suitable electrically insulative material, such as fluorinated ethylene propylene (FEP) or polytetrafluoroethylene (PTFE). In the illustrated embodiment, the sheath 46 is affixed to the shaft 38, and may be applied to the shaft 38 using any suitable means. For example, the sheath 46 can be applied to the shaft 38 as a heat shrink or can be extruded onto the shaft 38. Alternatively, the sheath 46 may be pre-formed and affixed to the shaft 38 by sliding the sheath 46 over the shaft 38 (or sliding the shaft 38 through the sheath 46) and locking the shaft 38/sheath 46 relative to each other using suitable means, such as pin screws.

A portion of the shaft 38 is exposed, such that a tip electrode 48 is formed at the distal end 42 of the shaft 38. In the embodiment illustrated in FIG. 2, the sheath 46 extends the entire length of the shaft 38, so that the tip electrode 48 is confined to a distal-facing surface of the shaft 38. In this manner, RF energy will be conveyed primarily in the distal direction, so that solid tissue located immediately axial to the tip electrode 48 may be electrosurgically ablated to create a channel through which the shaft 38 passes without substantially ablating the solid tissue immediately radial to the tip electrode 48. Thus, although the distal tip of the percutaneous probe 18 is atraumatic, rapid advancement of the percutaneous probe 18 through solid tissue is facilitated without causing collateral damage to tissue radial to the percutaneous probe 18.

Alternatively, as shown in FIG. 4, a radial portion of the distal end 42 of the shaft 38 may be exposed to form a tip electrode 48 as long as the RF energy radially conveyed from the tip electrode 48, based on the anticipated power level of the RF energy conveyed by the RF generator 14, does not substantially ablate the tissue immediately radial to the tip electrode 48 as the percutaneous probe 18 is advanced through the tissue.

For the purposes of this specification, an electrode is configured for not substantially ablating tissue immediately radial to the tip electrode, if the tip electrode, when energized, conveys electrical energy that is greater in the axial direction than in the radial direction. In this manner, very little tissue ablation will occur immediately radial to the tip electrode as it is axially pushed through the tissue. For example, it is desirable that less than 1 mm, and preferable that less than 0.1 mm, of the tissue radial to the tip electrode be ablated as the tip electrode is axially moved through the tissue. Ultimately, the amount of radial tissue ablation should be minimized, such that the percutaneous probe 18 may easily pass through the tissue. For example, such tissue ablation may result in a channel having a diameter equal to the diameter of the percutaneous probe 18, including the sheath 46, or may even result in a channel having a diameter less than the percutaneous probe 18, in which case, the channel will be dilated by the sheath 46 as the percutaneous probe 18 is advanced through the channel. It has been discovered during various pathological tests that any radial ablation created from a tip electrode such as that illustrated in FIG. 3 is undetectable to the naked eye when viewing a cross-section of tissue through which the tip electrode has been percutaneous introduced.

While the tip electrodes 48, 50 are shown in FIGS. 3 and 4 as being cylindrical in shape, the tip electrodes may vary in shape. For example, the distal end 42 of the shaft 38 illustrated in FIG. 5 is shaped to form a hemispherical tip electrode 52. In this manner, the drag between the tip electrode 52 and the tissue is reduced, while facilitating any necessary dilation of the channel, as the percutaneous probe 18 is advanced through the channel. As another example, the distal end 42 of the shaft 38 illustrated in FIG. 6 is shaped to from a spherical tip electrode 53. While the entire surface of the distal tip of the shaft 38 is left exposed, the maximum current density will tend to be at the distal surface of the tip electrode 53, thereby inducing tissue ablation in the distal direction, while preventing, or otherwise minimizing, tissue ablation in the radial direction.

It should be noted that, due to the small profile of the afore-described electrodes, and the fact that the delivered power density falls off inversely as the fourth power of the distance from the electrodes, the thermal effects of the passage of the percutaneous probe 18 through solid tissue is minimal. Thus, the patient should experience no greater physical pain than would otherwise be experienced when conventional needles are used. In addition, the surface of the channel created through the tissue by the ablation energy distally conveyed from the tip electrode 48 may be coagulated, thereby minimizing or preventing blood loss.

Referring back to FIG. 1, the percutaneous probe 18 further comprises a handle 55 and an electrical connector 54 carried by the handle 55. The handle 55 is preferably composed of a durable and rigid material, such as medical grade plastic, and is ergonomically molded to allow a physician to more easily manipulate the percutaneous probe. The handle 55 may be mounted to the proximal end 40 of the shaft 38 using any suitable fastening means, e.g., gluing or a compression fit. The electrical connector 54 is electrically coupled to the tip electrode 48 (or alternatively the tip electrodes 50, 52, 53) via the electrically conductive shaft 38. The electrical connector 54 is adapted to removably mate with the RF cable 16 connected to the RF generator 14. Alternatively, the RF cable 16 may be hardwired into the electrical connector 54. The percutaneous probe 18 optionally comprises an RF activation button 57 carried by the handle 55. Actuation of the RF activation button 57 controls the flow of RF energy delivered from the electrical connector 54 to the tip electrode 48.

In the illustrated embodiment, the percutaneous probe 18 comprises a sensor capable of sensing physiological information, such as the impedance, temperature, or pressure, of the tissue adjacent the tip electrode 48. In the case where tissue impedance is to be measured, the tip electrode 48, itself, will be the impedance sensor. In the case where temperature or pressure is to be measured, a separate temperature sensor, such as a thermistor or thermocouple, or a separate pressure sensor, may be carried by the distal end of the percutaneous probe 18 adjacent the tip electrode 48. The measurement of physiological parameters adjacent the tip electrode 48 will allow the nature of the tissue in which the tip electrode 48 is located to be determined.

Referring back to FIG. 1, the RF generator 14 is electrically connected to the electrical connector 54 of percutaneous probe 18 via the cable 16. The RF generator 14 may be a conventional RF power supply that operates at a frequency in the range from 300 KHz to 9.5 MHz, with a conventional sinusoidal or non-sinusoidal wave form. Such power supplies are available from many commercial suppliers, such as Valleylab, Aspen, and Bovie. Most general purpose electrosurgical power supplies, however, operate at higher voltages and powers than would normally be necessary or suitable for tissue ablation. Thus, such power supplies would usually be operated at the lower ends of their voltage and power capabilities.

More suitable power supplies will be capable of supplying an ablation current at a relatively low voltage, typically below 150V (peak-to-peak), usually being from 50V to 100V. The power will usually be from 5 W to 200 W, usually having a sine wave form, although other wave forms would also be acceptable. Alternatively, the RF energy may be pulsed (e.g., each pulse may have a duration of 0.2 s), which has been shown to provide a more efficient tissue ablation. In general, the amount of power necessary to allow rapid advancement of the percutaneous probe 18 through tissue will be dictated by the size of the tip electrode 48 (or alternatively tip electrodes 50, 52, 53). Because the size of the tip electrode 48 is relatively small, the amount of power necessary to provide this effect will be relatively low. While power at a level of 50 W will be almost always be sufficient, in most cases power at a level of 30 W, and oftentimes power at a level of 10 W, will be sufficient to allow rapid advancement of the percutaneous probe 18. Preferably, the RF generator 14 is operated at a power level within the range 1 W-50 W, and more preferably, within the range of 5 W-30 W, so that the percutaneous probe 18 can be rapidly advanced through solid tissue without causing excessive collateral damage to the tissue.

Power supplies capable of operating within these ranges are available from commercial vendors, such as Boston Scientific Corporation of San Jose, Calif., who markets these power supplies under the trademarks RF2000 (100 W) and RF3000 (200 W). Notably, these power supplies have built-in impedance and temperature measurement circuitry that may operate with impedance or temperature sensors located on the distal end of the percutaneous probe 18.

In the illustrated embodiment, RF current is delivered from the RF generator 14 to the tip electrode 48 (or alternatively the tip electrodes 50, 52, 53) of the percutaneous probe 18 in a monopolar fashion, which means that current will pass from the tip electrode 48, which is configured to concentrate the energy flux in order to have an injurious effect on the distally adjacent tissue, and a dispersive electrode (not shown), which is located remotely from the tip electrode 48 and has a sufficiently large area (typically 130 cm² for an adult), so that the current density is low and non-injurious to surrounding tissue. The dispersive electrode may be attached externally to the patient, e.g., using a contact pad placed on the patient's flank. In the case where impedance is to be measured, the impedance between the tip electrode 48 and the indifferent patch electrode may be measured by the RF generator 14.

While the percutaneous probe 18 has been described as being a single integrated device, percutaneous probes constructed in accordance with the present inventions can comprise multiple devices that interact with each other to provide a percutaneous probe with an atraumatic distal tip.

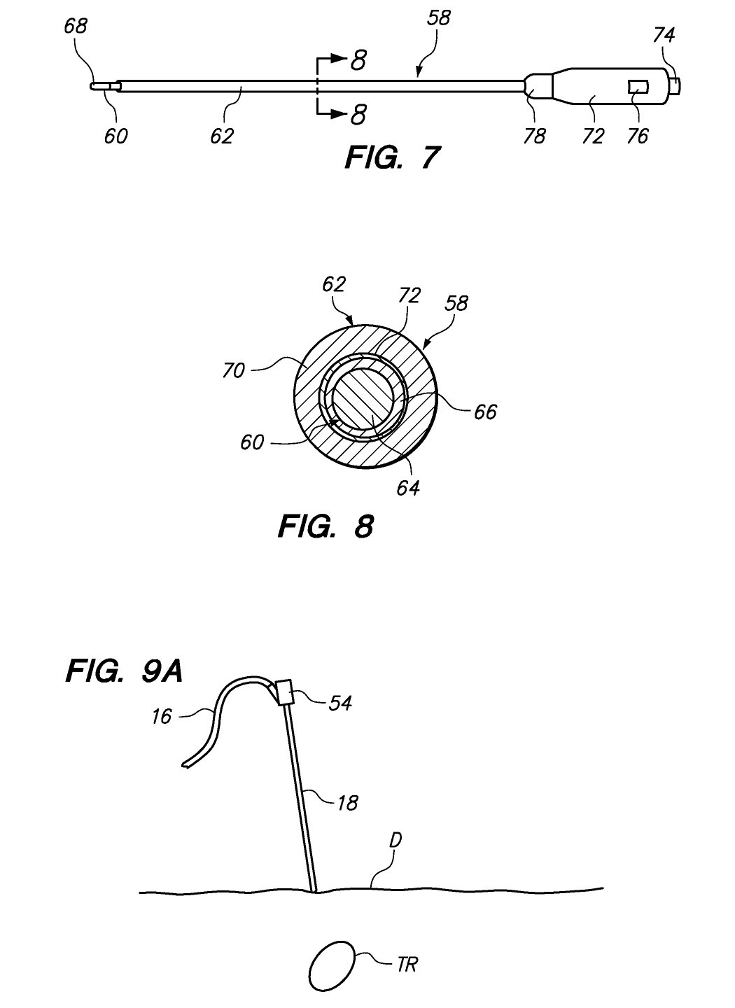

For example, FIGS. 7 and 8 illustrate an alternative embodiment of a percutaneous probe 58 that includes an inner member 60 and a cannula 62 through which the inner member 60 can be disposed. The inner member 60 is similar to the previously described percutaneous probe 18 in that it includes an elongated, electrically conductive, shaft 64 and a sheath 66 disposed over the shaft 64 to create a distal tip electrode 68 at the distal end of the shaft 64. As with the percutaneous probe 18, RF energy will be conveyed from the distal tip electrode 68 primarily in the distal direction, so that solid tissue located immediately axial to the tip electrode 68 may be electrosurgically ablated to create a channel through which the shaft 64 passes without substantially ablating the solid tissue immediately radial to the tip electrode 68. The tip electrode 68 may be similar to, e.g., the tip electrodes illustrated in FIGS. 2 and 4-6. The inner member 60 differs from the percutaneous probe 18 in that the shaft 64 is flexible and sized such that it is capable of being introduced into the cannula 62. For example, the shaft 64 may be sized such that the diameter of the inner member 60 is within the range 0.004″-0.030″.

The cannula 62 comprises an elongated cannula shaft 70 and a lumen 72 extending through the cannula shaft 70. To provide the necessary axial strength for the percutaneous probe 58, the cannula shaft 70 is substantially more rigid than the inner member shaft 64. For example, the cannula shaft 70 may have a wall thickness and may be composed of a material similar to those used to manufacture conventional needles, e.g., stainless steel. The length of the inner member shaft 64 is slightly greater than that of the cannula shaft 70, so that the distal tip electrode 68 extends from the distal end of the cannula shaft 70. For example, in the illustrated embodiment, the distal tip electrode 68 may extend 1/16″ from the distal end of the cannula shaft 70. In this manner, as RF energy is delivered to the distal tip electrode 68 of the percutaneous probe 58, the cannula 62 may be percutaneously introduced through solid tissue.

The inner member 60 includes a handle 72 and an electrical connector 74 carried by the handle 72. The handle 72 is preferably composed of a durable and rigid material, such as medical grade plastic, and is ergonomically molded to allow a physician to more easily manipulate the percutaneous probe. The electrical connector 74 is electrically coupled to the tip electrode 68 via the electrically conductive shaft 64. The electrical connector 74 is adapted to removably mate with the RF cable 16 connected to the RF generator 14. Alternatively, the RF cable 16 may be hardwired into the electrical connector 74. The inner member 60 optionally comprises an RF activation button 76 carried by the handle 72. Actuation of the RF activation button 76 controls the flow of RF energy delivered from the electrical connector 74 to the tip electrode 68. The percutaneous probe 18 may optionally carry a physiological sensor, which may be the tip electrode 68 if tissue impedance is to be measured, or a separate sensor if temperature or pressure is to be measured.

The inner member 60 and cannula 62 may be mated together in a locking arrangement to facilitate the percutaneous introduction thereof as a single integrated device. To this end, the cannula 62 includes a proximal adapter 78 suitably mounted on the proximal end of the cannula shaft 70. In the illustrated embodiment, the handle 72 and proximal adapter 78 have respective male and female portions that removably mate with each other.

Having described the structure of the catheter introducer system 10, its operation in providing an initial pathway into the body of a patient to facilitate percutaneous insertion of a catheter for draining excess fluid accumulated in a tissue region, such as a wound, sore, or body cavity to prevent the build-up of fluid in the patient or to prepare for surgery, will now be described. As examples, fluid may be drained from an abscess (an infected fluid collection), cyst (a non-infected fluid collection, hematoma (a collection of blood), biloma (collection of bile), or urinoma (a collection of urine). Introduction of the components of the catheter introducer system 10, as well as the catheter to be introduced, can be visualized using conventional imaging modalities, such as computed tomography (CT), ultrasound, or fluoroscopy.

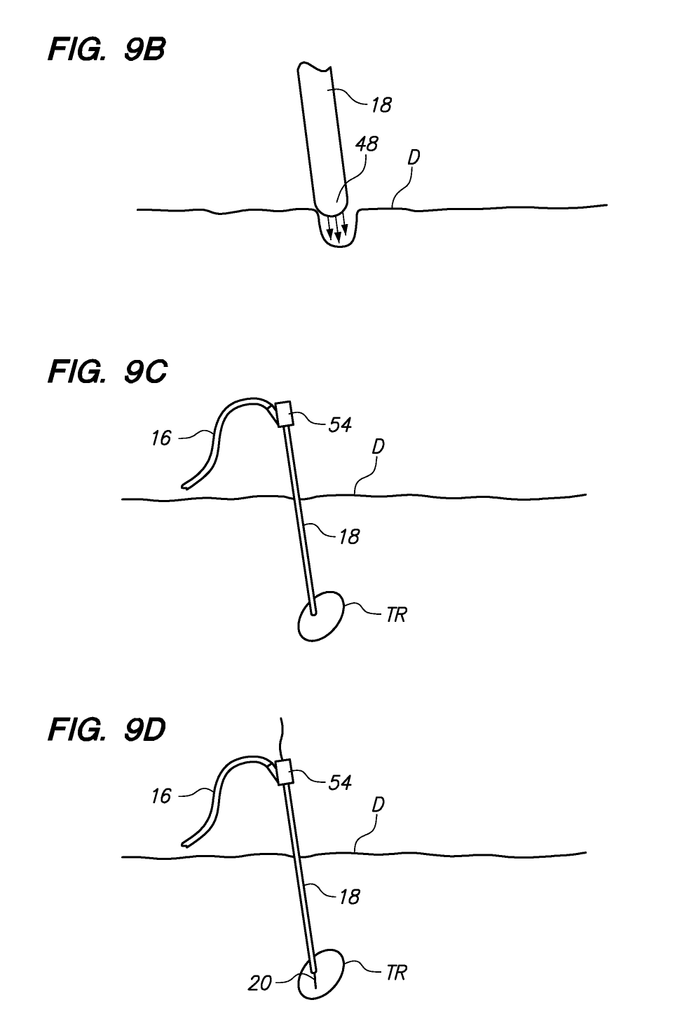

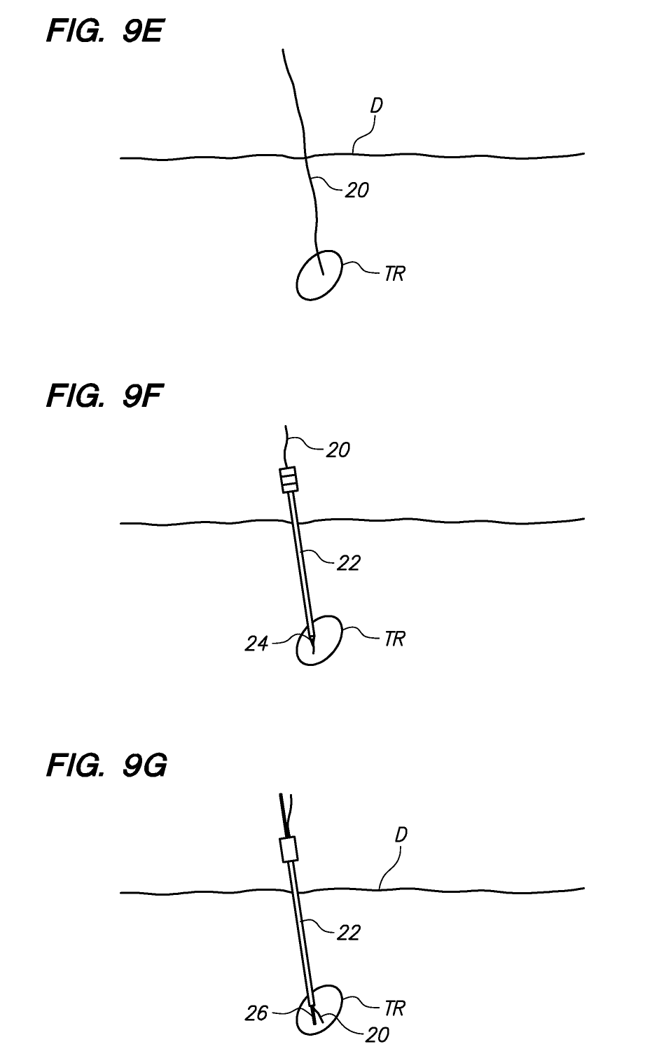

Referring to FIGS. 9A-9H, the operation of the catheter introducer system 10 is described in percutaneously introducing a catheter C (shown in FIG. 9H) into a patient to drain fluid from a fluid-filled tissue region TR located beneath the derma D. First, the RF generator 14 (shown in FIG. 1) is coupled to the connector 54 of the percutaneous probe 18 via RF cable 16, and the atraumatic distal tip of the percutaneous probe 18 is placed against the derma D of the patient (FIG. 9A). Alternatively, if the percutaneous probe 18 is used, the RF generator 14 is coupled to the connector 74 of the inner member 60. In either case, absent the application of RF energy, a moderate amount of axial pressure may be applied to the percutaneous probe 18 without puncturing the derma D.

Next, while applying a moderate amount of axial pressure to the percutaneous probe 18 (or alternatively, the percutaneous probe 58 with the inner member 60 and cannula 62 mated with each other), the RF activation button 57 (or alternatively, the RF actuation button 76) (shown in FIG. 1) is actuated to convey the RF energy delivered from the RF generator 14 through the percutaneous probe 18, and distally out of the tip electrode 48 (shown by arrows), thereby ablating tissue immediately axial to the tip electrode 48 (FIG. 9B). As a result, the distal tip of the percutaneous probe 18 readily advances along the channel created by the ablation through the tissue layers, including the dermal tissue layers, fat, and muscle, and into the tissue region TR (FIG. 9C).

If the percutaneous probe 18 (or alternatively, the percutaneous probe 58) carries a sensor, a physiological parameter, such as impedance, temperature, or pressure, can be monitored to ensure that the distal tip of the percutaneous probe 18 is properly located within the treatment region TR. For example, if the tip electrode 48 is used as an impedance sensor, the impedance between it and the indifferent patch electrode may be measured to detect an impedance change as the tip electrode 48 enters the treatment region TR. That is, once the tip electrode 48 enters the fluid-filled tissue region TR, the measured impedance will conspicuously drop, thereby providing an indication that the tip electrode 48 has entered the tissue region TR, and further advancement of the percutaneous probe 18 is unnecessary. If a separate temperature sensor and/or a separate pressure sensor is utilized, the measured temperature and/or measured pressure will conspicuously increase as the tip electrode 48 enters the fluid filled tissue region TR, thereby providing an indication that the tip electrode 48 has entered the tissue region TR, and further advancement of the percutaneous probe 18 is unnecessary.

Once the tip electrode 48 is within tissue region TR, the safety guidewire 20 is introduced through the lumen 44 of the percutaneous probe 18 and advanced out of the distal tip of the percutaneous probe 18 into the tissue region TR (FIG. 9D). If the percutaneous probe 58 is used, the inner member 60 is unmated and removed from the cannula 62, and the guidewire 20 is introduced through the lumen 72 of the cannula 62 and advanced out of the distal tip of the cannula 62 into the tissue region TR.

Next, the percutaneous probe 18 (or alternatively, the cannula 62 of the percutaneous probe 58) is removed from the patient, leaving the safety guidewire 20 in place (FIG. 9E). Because the surface of the channel created by the percutaneous probe 18 is coagulated by the ablation energy, blood loss through the channel may be minimized or prevented when the percutaneous probe 18 is removed. Notably, the relatively thin guidewire 20 allows the lumen 44, and thus, the percutaneous probe 18, to have a low profile, thereby minimizing the pain and tissue trauma suffered by the patient. However, because the relatively thin guidewire 20 is not stiff enough to properly place the flexible catheter C, the larger working guidewire 26 must be used.

To this end, the locked combination of the introducer cannula sheath 22 and dilator/catheter assembly 24 is then introduced over the safety guidewire 20 until the distal tip of the introducer cannula sheath 22 is located within the tissue region TR (FIG. 9F). The distal tip of the dilator 28 and the rigidity of the cannula 30 facilitate advancement of the otherwise flexible introducer cannula sheath 22 through the various tissue layers. The optional radiopaque marker on the distal end of the introducer cannula sheath 22 may be monitored to ensure that the introducer cannula sheath 22 is within the tissue region TR.

Next, the dilator/catheter assembly 24 is removed from the patient, leaving the introducer cannula sheath 22 in place, and then the working guidewire 26 is introduced through the lumen of the introducer cannula sheath 22 alongside the safety guidewire 20, and advanced out of the distal tip of the introducer cannula sheath 22 into the tissue region TR (FIG. 9G). The introducer cannula sheath 22 and safety guidewire 20 are removed from the patient, leaving the working guidewire 26 in place, and the catheter C is advanced over the working guidewire 26 into the tissue region TR (FIG. 9H). The working guidewire 26 may then be removed from the catheter C, and fluid drained from the tissue region TR via the catheter C.

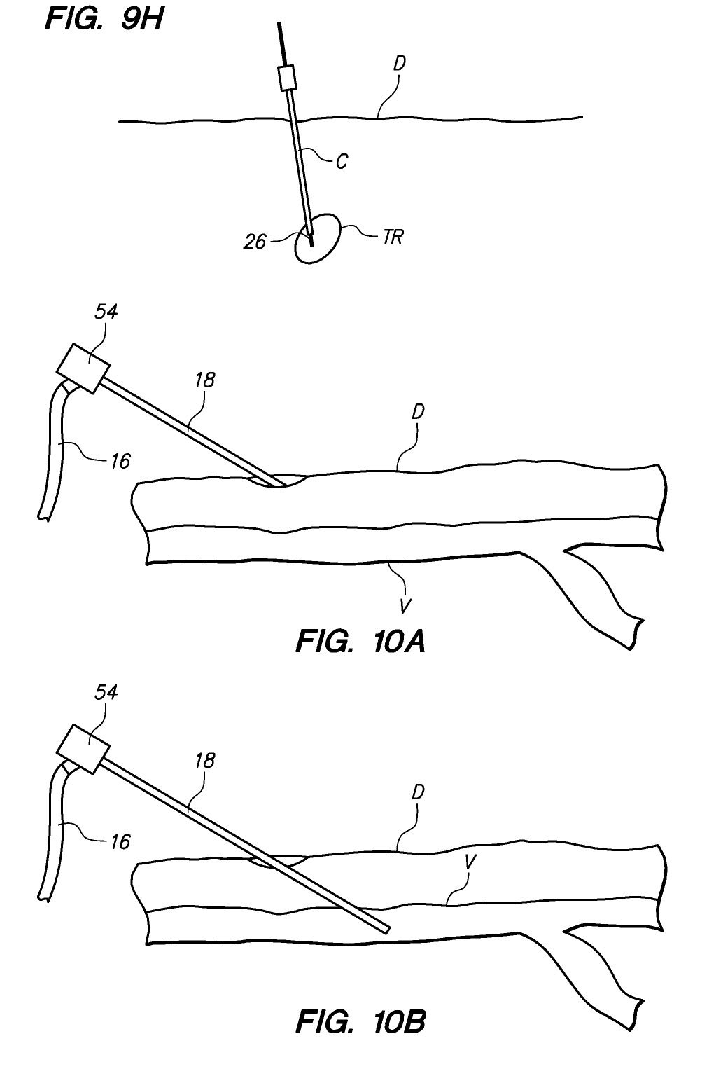

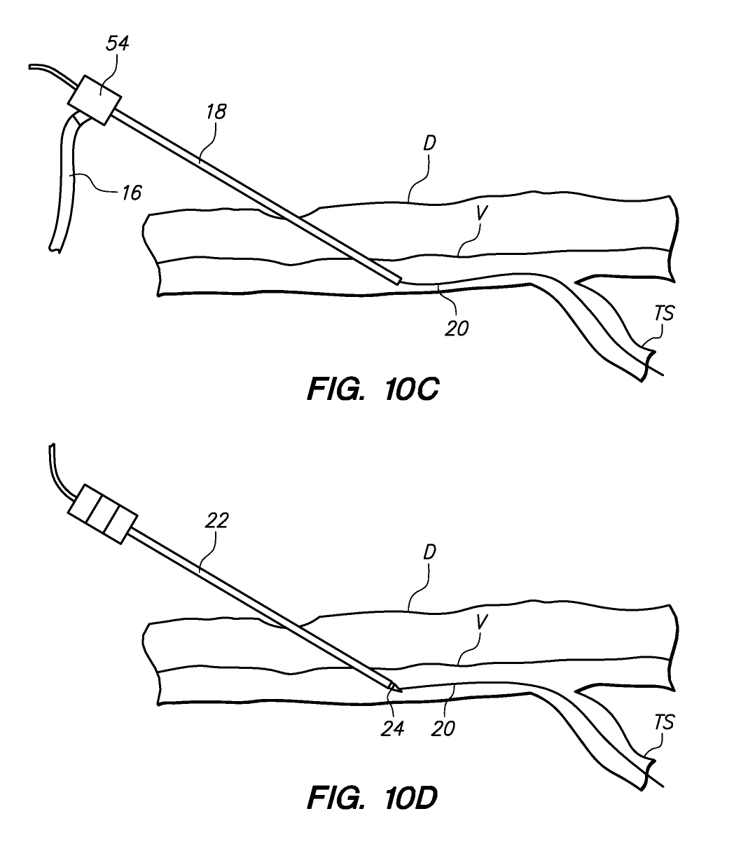

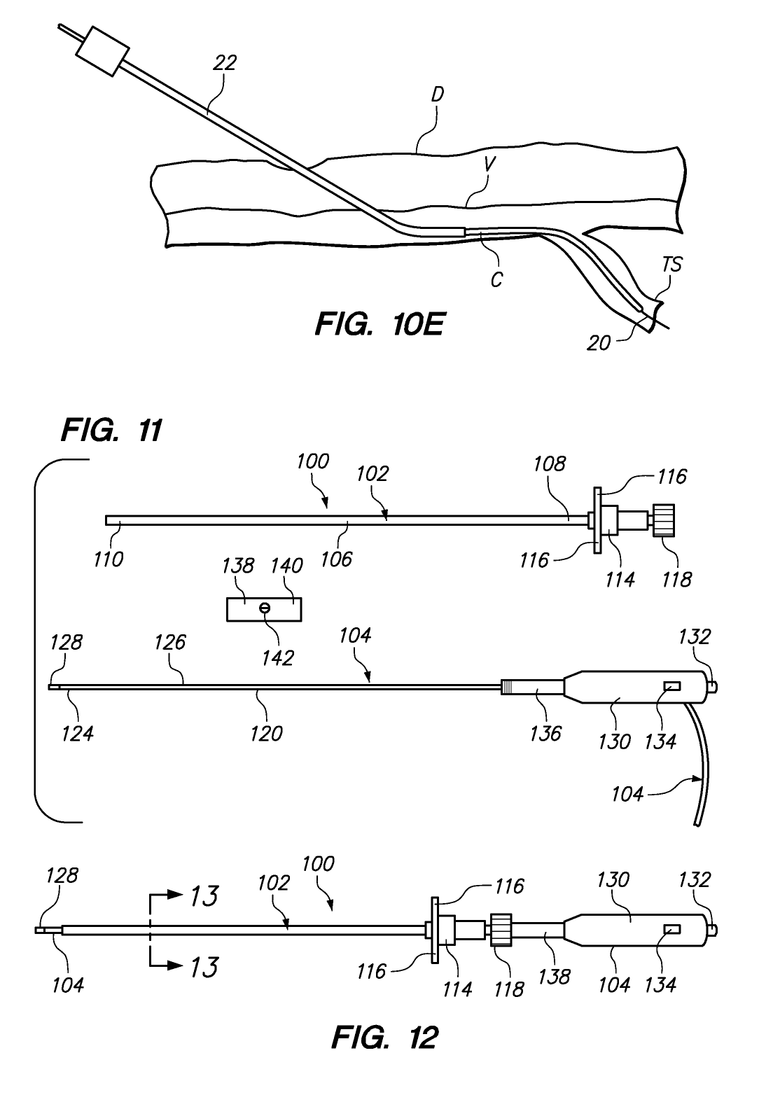

While the percutaneous probe 18 has been described as facilitating percutaneous access for catheters to an extravascular tissue region, the percutaneous probe 18 can be used to provide percutaneous access for catheters to an intravascular tissue region. For example, as illustrated in FIGS. 10A-10E, the catheter introducer system 10 can be used to percutaneously introduce a catheter C into the vasculature of a patient via an entry blood vessel V, such as the femoral artery of the patient, located beneath the derma D. In this case, only a single guidewire, and in particular, the guidewire 20 is used.

First, the RF generator 14 is coupled to the connector 54 of the percutaneous probe 18 via RF cable 16, and the atraumatic distal tip of the percutaneous probe 18 is placed against the derma D of the patient (FIG. 10A). Significantly, absent the application of RF energy, a moderate amount of axial pressure may be applied to the percutaneous probe 18 without puncturing the derma D.

Next, while applying a moderate amount of axial pressure to the percutaneous probe 18, the RF activation button 57 (shown in FIG. 1) is actuated to convey the RF energy delivered from the RF generator 14 through the percutaneous probe 18, and distally out of the tip electrode 48 (shown by arrows), thereby ablating tissue immediately axial to the tip electrode 48. As a result, the distal tip of the percutaneous probe 18 readily advances along the channel created by the ablation through the tissue layers, including the dermal tissue layers, fat, and muscle, and into the entry blood vessel V (FIG. 10B).

If the percutaneous probe 18 carries a sensor, a physiological parameter, such as impedance, temperature, or pressure, can be monitored to ensure that the distal tip of the percutaneous probe 18 is properly located within the blood vessel V. For example, if the tip electrode 48 is used as an impedance sensor, the impedance between it and the indifferent patch electrode may be measured to detect an impedance change as the tip electrode 48 enters the blood pool within the blood vessel V. That is, once the tip electrode 48 enters the blood pool, the measured impedance will conspicuously drop, thereby providing an indication that the tip electrode 48 has entered the blood vessel V, and further advancement of the percutaneous probe 18 is unnecessary. If a separate temperature sensor and/or a separate pressure sensor is utilized, the measured temperature and/or measured pressure will conspicuously change as the tip electrode 48 enters the blood pool, thereby providing an indication that the tip electrode 48 has entered the blood vessel V, and further advancement of the percutaneous probe 18 is unnecessary. For example, if the blood vessel V is an artery, which carries warm oxygenated blood, the temperature and pressure may increase, and if the blood vessel V is a vein, which carries relatively cool deoxygenated blood, the pressure may increase, but the temperature may decrease.

Once the tip electrode 48 is within the blood vessel V, the guidewire 20 is introduced into through the lumen 44 of the percutaneous probe 18 and advanced out of the distal tip of the percutaneous probe 18 into the blood vessel V, where it can be further advanced through the vasculature of the patient to a target site TS (FIG. 1C). Next, the percutaneous probe 18 is removed from the patient, leaving the guidewire 20 in place, and then the locked combination of the introducer cannula sheath 22 and dilator/catheter assembly 24 is introduced over the guidewire 20 until the distal tip of the introducer cannula sheath 22 is located within the blood vessel V (FIG. 10D). Because the surface of the channel created by the percutaneous probe 18 is coagulated by the ablation energy, blood loss through the channel may be minimized or prevented when the percutaneous probe 18 is removed. The distal tip of the dilator 28 and the rigidity of the cannula 30 facilitate advancement of the otherwise flexible introducer cannula sheath 22 through the various tissue layers. The optional radiopaque marker (not shown) on the distal end of the introducer cannula sheath 22 may be monitored to ensure that the introducer cannula sheath 22 is well within the blood vessel V.

Next, the dilator/catheter assembly 24 is removed from the patient, leaving the guidewire 20 and introducer cannula sheath 22 in place, and the catheter C is advanced over the guidewire 20, through the introducer cannula sheath 22, and into the blood vessel V, where it can be further advanced over the guidewire 20 to the target site TS (FIG. 10E).

While the percutaneous probes 18, 58 have been described in conjunction with guidewires and introducers to introduce catheters within a patient, RF-activated probes with atraumatic tips can be used as the guidewires themselves and/or as a means to directly introduce catheters into the patient without the use of additional guidewires/introducers. For example, FIGS. 11-13 illustrate a catheter assembly 100 constructed in accordance with another embodiment of the present invention. The catheter assembly 100 generally comprises a catheter 102 and an RF-activated guidewire 104 that can be inserted within the catheter 102 to facilitate the percutaneous introduction of the catheter 102 through the derma of a patient.

The catheter 102 may be any conventional diagnostic or therapeutic catheter that measures a physiological parameter of a patient or provides a therapeutic effect. For the purpose of this specification, a diagnostic or therapeutic catheter does not include a guide sheath or catheter that merely provides the function of guiding other devices within a patient. As illustrated in FIG. 11, the catheter 102 includes an elongated catheter shaft 106 having a proximal end 108 and a distal end 110.

The catheter shaft 106 may have a standard size, e.g., a length in the range of 2-20 cm, typically in the range of 5-15 cm, and a diameter in the range of 5 F-10 F, typically in the range of 6 F-8 F. The catheter shaft 106 may be manufactured in a conventional manner and may be composed of a suitable biocompatible material that imparts the desired flexibility to the catheter 102 to facilitate its advancement through the vasculature of a patient. The catheter 102 further includes a guidewire lumen 112 axially extending through the catheter shaft 106. The diameter of the guidewire lumen 112 is sized to conform to the outer diameter of the guidewire 104. The catheter 102 further comprises a proximal adapter 114 mounted to the proximal end of the catheter shaft 106 using any suitable fastening means, e.g., gluing or a compression fit. In the illustrated embodiment, the proximal adapter 114 has winged protrusions 116 to prevent interference with the patient's skin. The proximal adapter 114 further comprises a mechanical connector 118, e.g., a luer connector.

The guidewire 104 is similar to the previously described percutaneous probe 18 in that it includes an elongated, electrically conductive shaft 120 having a proximal end 122 and a distal end 124, and an electrically insulative sheath 126 disposed over the shaft 120 to create a distal tip electrode 128 at the distal end 124 of the shaft 120. As with the percutaneous probe 18, RF energy will be conveyed from the distal tip electrode 128 primarily in the distal direction, so that solid tissue located immediately axial to the tip electrode 128 may be electrosurgically ablated to create a channel through which the shaft 120 passes without substantially ablating the solid tissue immediately radial to the tip electrode 128. The tip electrode 128 may be similar to, e.g., the tip electrodes illustrated in FIGS. 2 and 4-6. The guidewire 104 differs from the percutaneous probe 18 in that the shaft 120 is flexible and sized such that it is capable of being introduced into the catheter 102. For example, the guidewire shaft 120 may be sized such that the diameter of the guidewire 104 is within the range 0.004″-0.030″.

The length of the guidewire shaft 120 is greater than that of the catheter shaft 106, so that the distal tip electrode 128 extends from the distal end 110 of the catheter shaft 106 when the guidewire 104 is inserted into the catheter 102 (as shown in FIG. 12). For example, in the illustrated embodiment, the distal tip electrode 128 may extend 1/16″ from the distal end of the catheter shaft 106. In this manner, as RF energy is delivered to the distal tip electrode 124 of the guidewire 104, the catheter 102 may be percutaneously introduced through solid tissue. Preferably, the length of the guidewire 104 is at least 6-8 inches greater than that of the catheter shaft 106, so that the guidewire 104 can provide a mechanical guiding function for advancing the catheter 102 through the vasculature of a patient. That is, the guidewire 104 may be maneuvered through a blood vessel in advance of the catheter 102.

The catheter assembly 100 further comprises a handle 130 and an electrical connector 132 carried by the handle 130. The handle 130 is preferably composed of a durable and rigid material, such as medical grade plastic, and is ergonomically molded to allow a physician to more easily manipulate the percutaneous probe. The electrical connector 132 is electrically coupled to the tip electrode 128 via the electrically conductive shaft 120. The electrical connector 132 is adapted to removably mate with the RF cable 16 connected to the RF generator 14 (shown in FIG. 1). Alternatively, the RF cable 16 may be hardwired into the electrical connector 132. The catheter assembly 100 optionally comprises an RF activation button 134 carried by the handle 130. Actuation of the RF activation button 134 controls the flow of RF energy delivered from the electrical connector 132 to the tip electrode 128.

The handle 130 and proximal adapter 114 of the catheter 102 can be mated together in a locking arrangement to facilitate the percutaneous introduction of the catheter assembly 100 as a single integrated device. To this end, the handle 130 has a male connector portion 136 that removably mates with the connector 118 of the proximal adapter 114, e.g., using a threaded arrangement.

The handle 130, guidewire 104, and catheter 102 are arranged, such that the handle 130 is removably attached to the guidewire 104, and the guidewire 104 can be selectively affixed relative to the catheter 102 so that the length of the guidewire 104 extending from the catheter 102 can be adjusted. For example, during percutaneous introduction of the catheter assembly 100 into solid tissue, the length of the guidewire 104 extending from the catheter 100 may be set to be relatively small (e.g., 1/16″), and when the guidewire 104 is used to guide the catheter 102 through a blood vessel, the guidewire 104 may be freed from the assembly 100 so that it can be distally pushed, e.g., 6-8 inches, in advance of the catheter 102.

To this end, the guidewire 104 extends through the handle 130 into direct or indirect electrical contact with the electrical connector 132 and out of a port in the handle 130, so that it can be grasped and manipulated by a user. The guidewire 104 is slidably disposed within the handle 130, and thus, slidably disposed within the lumen 112 of the catheter 102. The catheter assembly 100 further comprises a locking mechanism 138, which in the illustrated embodiment, includes a collar 140 and a set screw 142. The collar 140 is sized to fit snugly over the connector portion 136 of the handle 130, and the set screw 142 is threadingly engaged with the collar 140 in a manner that allows it to extend through a hole (not shown) in the connector portion 136 of the handle 130 and engage the guidewire 104 in an interference arrangement. Thus, rotating the set screw 142 in one direction will release the guidewire 102, thereby allowing it to be slid relative to the catheter 102, and rotating the set screw 142 in an opposite direction will engage the guidewire 102, such that the guidewire 102 can be fixed relative to the catheter 102.

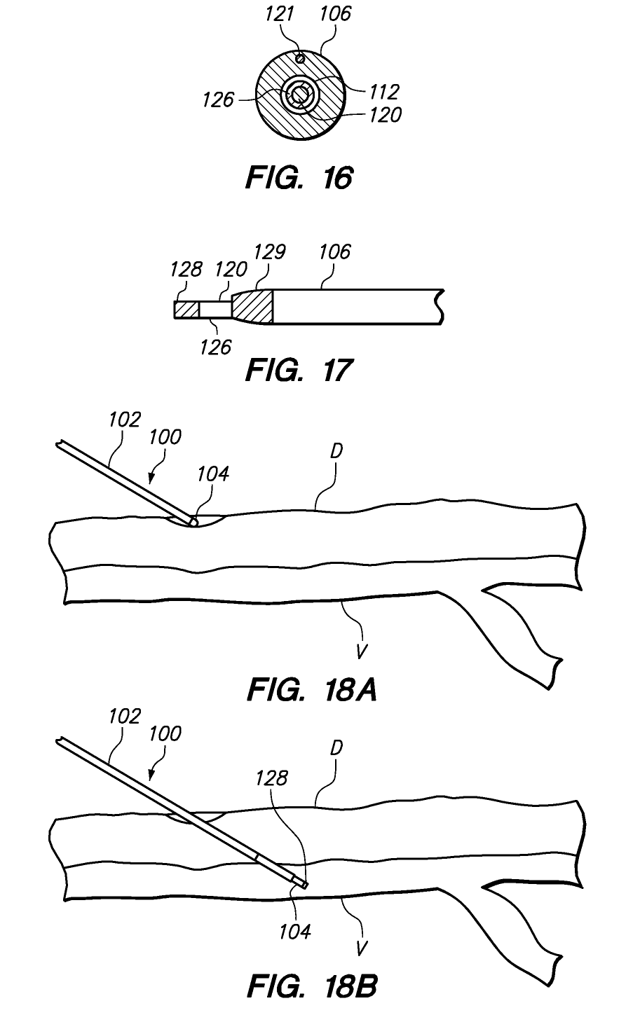

FIGS. 14-17 illustrate a catheter assembly 101 constructed in accordance with another embodiment of the present invention. The catheter assembly 101 is similar to the previously described catheter assembly 100, with the exception that the catheter assembly 101 may deliver electrical energy in a bipolar arrangement. In particular, the catheter 102 includes an electrode 129 disposed on the distal end 110 of the catheter shaft 106, and an electrical connector 133 disposed on the proximal adapter 114 of the catheter 102 and electrically coupled to the catheter electrode 129 via an RF wire 121 (shown in FIG. 16). The electrical connector 133 is adapted to removably mate with an RF cable (not shown) connected to the RF generator 14 (shown in FIG. 1). Alternatively, the RF cable may be hardwired into the electrical connector 133. As best seen in FIG. 17, the insulation 126 disposed over the guidewire shaft 120 electrically isolated the electrodes 128, 129 from each other.

Thus, RF current may be delivered between the tip electrode 128 and the catheter electrode 129, one of which will be the “positive” electrode, and the other of which will be a “negative” electrode. Notably, bipolar arrangements, which require the RF energy to traverse through a relatively small amount of tissue between the tightly spaced electrodes, are more efficient than monopolar arrangements, which require the RF energy to traverse through the thickness of the patient's body. As a result, bipolar electrodes ablate tissue more efficiently, thereby reducing the amount of power, reducing cauterization, and making percutaneous introduction of the catheter assembly 101 less painful. In addition, no grounding pads or electrodes are required.

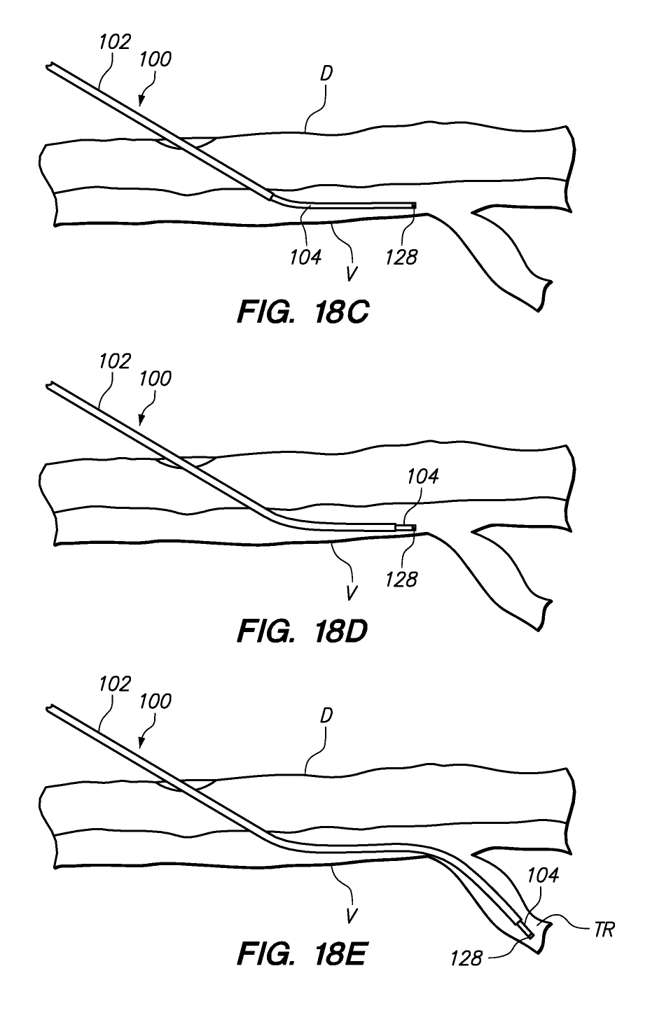

Having described the structure of the catheter introducer assembly 100, a method of percutaneously delivering the catheter introducer assembly 100 beneath the derma D of a patient into an entry blood vessel V, such as the femoral artery, with now be described with reference to FIGS. 18A-18E. While this method is described with respect to the catheter introducer assembly 100, the same method is applicable to the catheter introducer assembly 101.

First, if not already assembled, the handle 130 is mated to the proximal adapter 114 of the catheter 102, the guidewire 104 is threaded through the handle 130 and catheter lumen 112 until the tip electrode 128 extends from the distal end 124 of the catheter shaft 120 the desired distance, and the locking mechanism 138 is manipulated to axially fix the guidewire 104 relative to the catheter 102 by rotating the set screw 142 (see FIG. 12). Next, the RF generator 14 (shown in FIG. 1) is coupled to the connector 132 on the handle 130 via the RF cable 16 (and if the catheter assembly 101 is used, the RF generator 12 is coupled to the connector 133 on the proximal adapter 114 of the catheter 102 via another RF cable), and the atraumatic distal tip of the guidewire 104 is placed against the derma D of the patient (FIG. 18A). Significantly, absent the application of RF energy, a moderate amount of axial pressure may be applied to the catheter assembly 100 without puncturing the derma D.

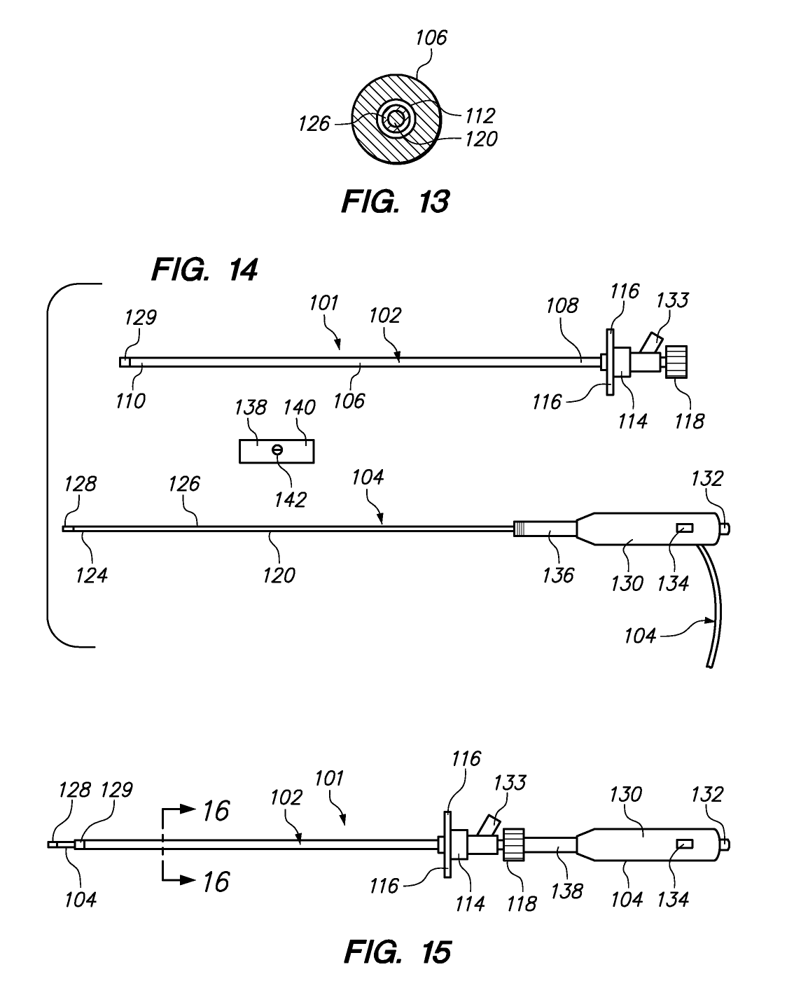

Next, while applying a moderate amount of axial pressure to the handle assembly 100, the RF activation button 134 (shown in FIG. 13) is actuated to convey the RF energy delivered from the RF generator 14 through the guidewire 104, and distally out of the tip electrode 128, thereby ablating tissue immediately axial to the tip electrode 128. As a result, the distal tip of the catheter assembly 100 readily advances along the channel created by the ablation through the tissue layers, including the dermal tissue layers, fat, and muscle, and into the entry blood vessel V (FIG. 18B). If the guidewire 104 carries a sensor, a physiological parameter, such as impedance, temperature, or pressure, can be monitored to ensure that the distal tip of the catheter assembly 100 is properly located within the blood vessel V, as described above with respect to the percutaneous probe 18.

Once the distal tip of the catheter assembly 100 is within the blood vessel V, the locking mechanism 138 is manipulated by rotating the set screw 142 in the opposite direction to axially release the guidewire 104, and the guidewire 104 is axially moved in the distal direction relative to the catheter 102 through the blood vessel V (FIG. 18C). Next, while the guidewire 104 is held in place, the catheter 102 is advanced along the guidewire 104 until the distal tip of the guidewire 104 is reached (FIG. 18D). The guidewire 104 and catheter 102 are advanced in this manner until the distal end 110 of the catheter shaft 106 reaches a target tissue site TS (FIG. 18E). The catheter 102 is then operated to perform the designated diagnostic and/or therapeutic function at the target tissue site TS.

While the RF-activated probes 18, 58, 104 have been described in conjunction with catheter introducer systems, it should be appreciated that RF-activated probes with atraumatic tips can be used in other applications. For example, an RF-activated probe may be used to percutaneously access tissue of a patient for delivering therapeutic agents, such as drugs, or for drawing fluid, such as blood, from the patient. An RF-activated probe may be used as a trocar to place laparoscopic and arthroscopic cannulae, or an RF-activated probe may be used in place of a Verres needle for accessing and insufflating the abdomen of a patient. Such RF-activated members would have a similar structure and be operated in a similar manner as the percutaneous probe 18 or percutaneous probe 58. As will be described in further detail below, an RF-activated probe can be used to introduce biopsy probes into a patient to obtain tissue samples.

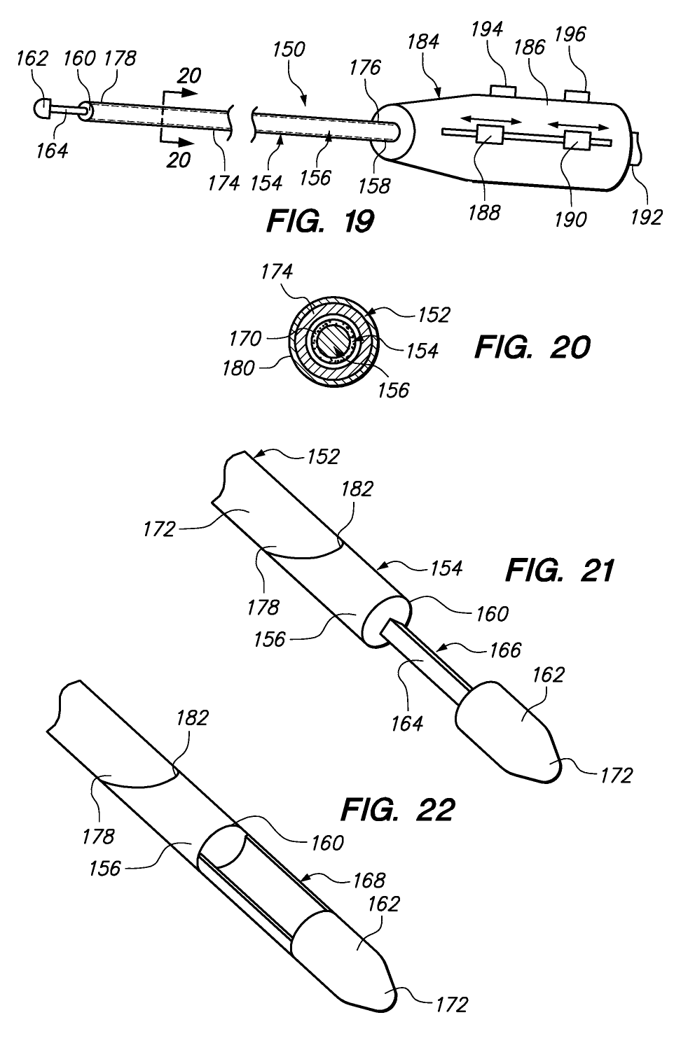

While the previously described RF-activated probes lend themselves well to providing percutaneous access into patients, RF-activated probes with atraumatic tips may also be used in other applications, such as biopsies. For example, FIGS. 19-21 illustrate a biopsy probe 150 constructed in accordance with another embodiment of the present invention. The biopsy probe 150 generally comprises an outer cannula 152 and an inner probe member 154 (shown in phantom in FIG. 19) that are axially movable relative to each other.

The inner probe member 154 comprises an elongated shaft 156 having a proximal end 158 and a distal end 160. As best illustrated in FIG. 21, the distal end 160 of the inner member shaft 156 forms an atraumatic distal tip 162, so that the biopsy probe 150 will not pierce the derma of the patient or healthcare worker when not energized with RF energy. The distal end 160 of the inner member shaft 156 also forms a proximally located tissue containment element 164. The containment element 164 takes the form of a reduced-diameter rod located at a lower region of the shaft 156, so that tissue in firm contact with the distal end of the biopsy probe 150 will prolapse into a space 166 above the containment element 164. In the illustrated embodiment, the cross-sectional geometry of the containment element 164 is triangular, but other cross-sectional geometries may be used. Biopsy probes with containment elements similar to that illustrated in FIG. 21 are marketed by Boston Scientific Corporation under the trademark Easy Core™. Alternatively, as illustrated in FIG. 22, the distal end 160 of the inner member shaft 156 has a containment element 168 that takes the form of a grooved notch, which allows a larger portion of tissue to prolapse. Biopsy probes with containment elements similar to that illustrated in FIG. 22 are marketed by Boston Scientific Corporation under the trademark ASAP™, and are described in U.S. Pat. No. 5,989,196, which is expressly incorporated herein by reference.

In the illustrated embodiment, the inner member shaft 156 is composed of a rigid, electrically conductive, material suitable to conduct electrical energy, such as RF energy, from the proximal end 158 to the distal end 160 thereof to effect an ablation function, as will be described in further detail below. As best shown in FIG. 20, the inner probe member 154 further comprises a sheath 170 disposed on the shaft 156. The sheath 170 is composed of a suitable electrically insulative material, such as fluorinated ethylene propylene (FEP) or polytetrafluoroethylene (PTFE). In the illustrated embodiment, the sheath 170 is affixed to the shaft 156, and may be applied to the shaft 156 using any suitable means. For example, the insulative sheath 170 can be applied to the shaft 156 as a heat shrink or can be extruded onto the shaft 156.

A portion of the inner member shaft 156 is exposed, such that a tip electrode 172 is formed at the distal tip 162 of the member shaft 156. In the embodiment illustrated in FIG. 19, the sheath 170 extends the length of the shaft 156 to a point just proximal to the tissue containment element 164, so that the tip electrode 172 is confined to the distal tip 162. As will be described below, the tissue containment element 164 will be retracted within the cannula 152, and will therefore, not form part of the tip electrode 172 during tissue ablation. Alternatively, the sheath 170 may extend over the tissue containment element 164 just proximal to the distal tip 162 of the shaft 156.

The cannula 152 comprises an elongated cannula shaft 174 having a proximal end 176 and a distal end 178, and a lumen 180 (shown in FIG. 20) extending through the cannula shaft 174. As can be appreciated, the inner probe member 154 is slidably disposed within the cannula lumen 180. The length of the inner member shaft 156 is slightly greater than that of the cannula shaft 174, so that the distal tip electrode 172 extends from the distal end of the cannula shaft 174 when the inner probe member 154 is fully retracted within the cannula 152. For example, in the illustrated embodiment, the distal tip electrode 172 may extend 1/16″ from the distal end of the cannula shaft 174. In this manner, as RF energy is delivered to the distal tip electrode 172, the biopsy probe 150 may be introduced through solid tissue. Thus, although the distal tip 162 of the inner member shaft 156 is atraumatic, rapid advancement of the biopsy probe 150 through solid tissue can be accomplished when RF energy is applied to the tip electrode 172.

Like the inner member shaft 156, the cannula shaft 174 is composed of a rigid, electrically conductive, material suitable to conduct electrical energy, such as RF energy, from the proximal end 176 to the distal end 178 thereof to effect an ablation function, as will be described in further detail below. The cannula 152 further comprises a sheath 180 disposed on the cannula shaft 174. The sheath 180 is composed of a suitable electrically insulative material, such as fluorinated ethylene propylene (FEP) or polytetrafluoroethylene (PTFE). In the illustrated embodiment, the sheath 180 is affixed to the cannula shaft 174, and may be applied to the shaft 174 using any suitable means. For example, the sheath 180 can be applied to the shaft 174 as a heat shrink or can be extruded onto the shaft 174.

A portion of the inner member shaft 156 is exposed, such that a cylindrical cutting electrode 182 is formed at the distal end 178 of the cannula shaft 174. Thus, when the cannula 152 is distally moved relative to the inner probe member 154, the distal edge of the cutting electrode 182 will shear off any tissue that has prolapsed into the space 166 adjacent the tissue containment element 164 of the inner probe member 154. Such tissue cutting function will be facilitated by the tissue ablation effected by the electrode 182. It can be appreciated that spring and firing mechanisms previously required to move a cannula and inner member relative to each other to effect tissue cutting is obviated by the use of ablation energy, resulting in a less complex biopsy probe that is easier to use.

While the cutting electrode 182 is illustrated as moving over the inner probe member 154 to effect the tissue cutting function, it should be appreciated that, in alternative embodiments, a cutting electrode may be movable within a cannula to effect the tissue cutting function. In this case, the cannula may have a side port through which tissue may prolapse, and the cutting electrode may be disposed on the distal end of an inner probe member slidably disposed within the cannula to shear off or cut tissue that has prolapsed through the side port in the cannula.

Referring back to FIG. 19, the biopsy probe 150 further comprises a handle assembly 184, which includes a handle 186, a pair of mechanical actuators 188, 190, and an electrical connector 192. The handle 186 is preferably composed of a durable and rigid material, such as medical grade plastic, and is ergonomically molded to allow a physician to more easily manipulate the percutaneous probe. The cannula 152 and inner probe member 154 are slidably mounted within the handle 186, such that they may coaxially and independently move relative to each other.

The mechanical actuators 188, 190 are respectively coupled to the inner probe member 154 and cannula 152 to effect the coaxial movement of these elements. In the illustrated embodiment, the mechanical actuators 188, 190 take the form of slide mechanisms that can be reciprocatably moved in the distal and proximal directions. That is, movement of the actuator 188 in the distal direction deploys the inner probe member 154 in the distal direction, and movement of the actuator 188 in the proximal direction retracts the inner probe member 154 in the proximal direction. Likewise, movement of the actuator 190 in the distal direction deploys the cannula 152 in the distal direction, and movement of the actuator 190 in the proximal direction retracts the cannula 152 in the proximal direction. The mechanical actuators 188, 190 may be coupled to the inner probe member 154 and respective cannula 152 using conventional techniques.

The electrical connector 192 is electrically coupled to the tip electrode 172 and cutting electrode 182 via the electrically conductive inner member shaft 156 and cannula shaft 174. The electrical connector 192 is adapted to removably mate with the RF cable 16 connected to the RF generator 14 (shown in FIG. 1). Alternatively, the RF cable 16 may be hardwired into the electrical connector 192. The handle assembly 184 optionally comprises a pair of RF activation buttons 194, 196 carried by the handle 186. Actuation of the RF activation button 194 controls the flow of RF energy delivered from the electrical connector 192 to the tip electrode 172, and actuation of the RF activation button 196 controls the flow of RF energy delivered from the electrical connector 192 to the cutting electrode 182.

In the illustrated embodiment, RF current is delivered from the RF generator 14 to the tip electrode 172 and cutting electrode 182 in a monopolar fashion. Alternatively, RF current may be delivered between the tip electrode 172 and cutting electrode 182, which means that current will pass between “positive” and “negative” electrodes. Bipolar arrangements, which require the RF energy to traverse through a relatively small amount of tissue between the tightly spaced electrodes, are more efficient than monopolar arrangements, which require the RF energy to traverse through the thickness of the patient's body. As a result, bipolar electrodes ablate tissue more efficiently.

Having described the structure of the biopsy probe 150, its operation in obtaining a sample of a tissue region, e.g., a tumor, within a patient will be described. In this method, the percutaneous probe 58 illustrated in FIGS. 7 and 8 is used as a coaxial introducer system for percutaneously introducing the biopsy probe 150 into the patient adjacent the tissue region. Placement of the percutaneous probe 58 within the patient and the subsequent introduction of the biopsy probe 150 can be visualized using conventional imaging modalities, such as computed tomography (CT), ultrasound, or fluoroscopy.

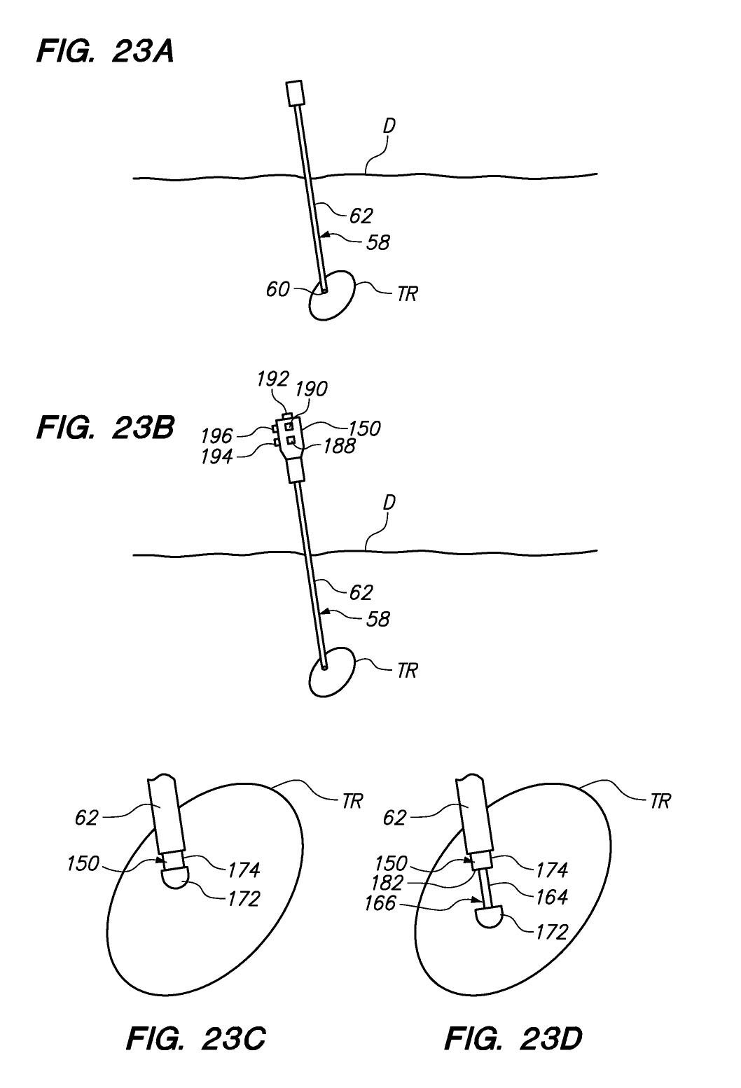

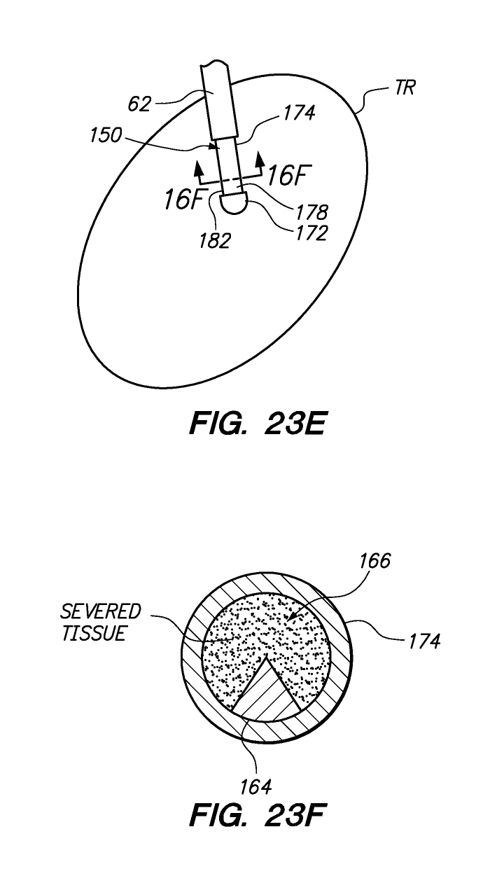

Referring to FIGS. 23A-23F, the percutaneous introduction of the biopsy probe 150 into a tissue region TR located beneath the derma D and the operation of the biopsy probe 150 to remove a sample from the tissue region TR will now be described. First, in the same manner described above in FIGS. 9A-9C, the percutaneous probe 58 of FIG. 7 is introduced through the derma D until the distal tip of the percutaneous probe 58 is adjacent the tissue region TR (FIG. 23A). Next, the inner member 60 is removed from the cannula 62 of the percutaneous probe 58, and, with the mechanical actuators 188, 190 moved to the most proximal positions to retract the cannula 154 and inner probe member 156, the biopsy probe 150 is introduced through the introducer cannula 62 into the patient (FIGS. 23B and 23C).

Next, the RF generator 14 (shown in FIG. 1) is coupled to the connector 192 of the biopsy probe 150 via RF cable 16, and as the RF activation button 194 is actuated to convey the RF energy delivered from the RF generator 14 through the inner member shaft 156 of the biopsy probe 150, and distally out of the tip electrode 172, thereby ablating tissue adjacent the tip electrode 172, the mechanical actuator 188 is slid forward in the distal direction. As a result, the inner probe member 154, and thus the tip electrode 172, readily advances into the tissue region TR, and a portion of the tissue region TR prolapses into the space 166 defined by the tissue containment element 164 of the inner probe member 154 (FIG. 23D).

Next, as the RF activation button 196 (shown in FIG. 23B) is actuated to convey the RF energy delivered from the RF generator 14 through the cannula shaft 174 of the biopsy probe 150, thereby ablating tissue adjacent the cutting electrode 182, the mechanical actuator 190 is slid forward in the distal direction. As a result, the cannula 152, and thus, the cutting electrode 182, readily advances through the tissue, thereby shearing or cutting the prolapsed tissue portion away from the remainder of the tissue region TR (FIGS. 23E and 23F). The biopsy probe 150 is then removed from the introducer cannula 152, and the severed tissue retained within the distal end 178 of the cannula shaft 174, removed from the biopsy probe 150 as a sample. The biopsy probe 150 may optionally be introduced through the introducer cannula 62, and by repeating the steps illustrated in FIGS. 23B-23F, can be operated again to obtain another tissue sample.

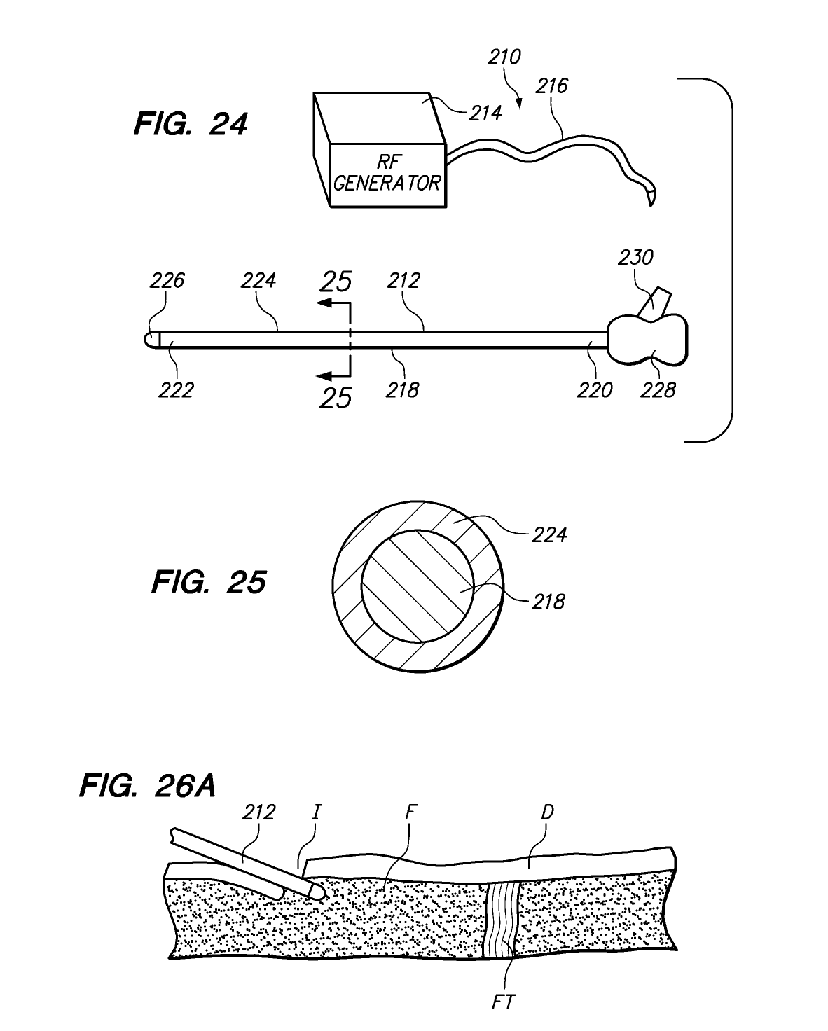

Referring to FIGS. 24 and 25, a subcutaneous tissue tunneling system 210 constructed in accordance with an embodiment of the present inventions will now be described. The tissue treatment system 210 generally comprises a RF-activated tunneling probe 212 and a tissue ablation source, and in particular a radio RF generator 214, configured for supplying RF energy to the tunneling probe 212 in a controlled manner via an RF cable 216.

The tunneling probe 212 has a suitable length, e.g., between 12 and 18 inches, and has a suitable outer diameter, e.g., between 1/16 inch and ½ inch. In the illustrated embodiment, the tunneling probe 212 is normally straight, but may alternatively be normally curved or be placed into a curved shape to facilitate tissue dissection. For example, the tunneling probe 212 may be curved to approximate the shape of an elongate structure that it is desired to dissect free of connective tissue, or to facilitate the maneuvering of the tunneling probe 212 around an obstruction.

The tunneling probe 212 generally comprises an elongated shaft 218 having a proximal end 220 and a distal end 222. The shaft 218 is composed of a rigid material, such as stainless steel, to provide adequate rigidity for the tunneling probe 212 to tunnel between tissue layers. Alternatively, the shaft 218 may be formed from a semi-rigid material to accommodate situations where it is desirable to navigate torturous passages within the patient. The material from which the shaft 218 is composed is also electrically conductive, e.g., stainless steel, in order to conduct electromagnetic energy, such as RF energy, from the proximal end 220 to the distal end 222 thereof to effect an ablation function, as will be described in further detail below. The distal end 222 of the shaft 218 is atraumatic, and in particular, a blunt tip 224 is formed at the distal end 222 of the shaft 218, so that the tunneling probe 212 does not inadvertently pierce through tissue outside of the path of the desired tunneling path. In the illustrated embodiment, the blunt tip 224 is olive-shaped, although other shapes providing a blunt surface are possible.

The tunneling probe 212 further comprises a sheath 226 disposed on the shaft 218. The sheath 226 is composed of a suitable electrically insulative material, such as FEP or PTFE. In the illustrated embodiment, the sheath 226 is affixed to the shaft 218, and may be applied to the shaft 218 using any suitable means. For example, the sheath 226 can be applied to the shaft 218 as a heat shrink or can be extruded onto the shaft 218. A portion of the shaft 218, and in particular the blunt distal tip 224, is exposed to form a tip electrode. Notably, the tip electrode 224, unlike the tip electrode 48 of the percutaneous probe 18, will convey RF energy in both the distal and radial directions. Because the channel made by the tunneling probe 212 may be larger than the diameter of the tunneling probe 212, radial tissue ablation can be tolerated.

The tunneling probe 212 further comprises a handle 228 and an electrical connector 230 carried by the handle 228. The handle 228 is preferably composed of a durable and rigid material, such as medical grade plastic, and is ergonomically molded to allow a physician to more easily manipulate the tunneling probe 212. The handle 228 may be mounted to the proximal end 220 of the shaft 218 of the tunneling probe 212 using any suitable fastening means, e.g., gluing or a compression fit. The electrical connector 230 is electrically coupled to the tip electrode 224 via the electrically conductive shaft 218. The electrical connector 230 is adapted to removably mate with the RF cable 216 connected to the RF generator 214. Alternatively, the RF cable 216 may be hardwired into the electrical connector 230.

In the illustrated embodiment, the tunneling probe 212 comprises a sensor capable of sensing physiological information, such as the impedance, temperature, or pressure, of the tissue adjacent the tip electrode 224. In the case where tissue impedance is to be measured, the tip electrode 224, itself, will be the impedance sensor. In the case where temperature or pressure is to be measured, a separate temperature sensor, such as a thermistor or thermocouple, or a separate pressure sensor, may be carried by the distal end of the tunneling probe 212 adjacent the tip electrode 224. Like with the previously described percutaneous probe 18, the measurement of physiological parameters adjacent the tip electrode 224 will allow the nature of the tissue in which the tip electrode 224 is located to be determined.

The RF generator 214 is electrically connected to the electrical connector 230 of the tunneling probe 212 via the cable 216. The RF generator 214 may be similar to the previously described RF generator 14. The only difference is that, due to the larger tip electrode 224, a greater amount of power necessary to effect tissue ablation may be required to allow rapid advancement of the tunneling probe 212 through fibrous tissue. For example, the amount of power may be 50 W or lower, and typically, 20 W or lower. Like the percutaneous probe 18, RF current may be delivered from the RF generator 214 to the tip electrode 224 of the tunneling probe 212 in a monopolar fashion.

Having described the structure of the tissue tunneling system 210, its operation in providing a tunnel subcutaneously within a patient's body will now be described. The tunnel may be made to accommodate various medical conduits, such as a catheter (e.g., a dialysis catheter) or an electrical lead (e.g., an extension between an implantable pulse generator (IPG) and a stimulation lead implanted within the patient's brain or adjacent the patient's spinal cord).

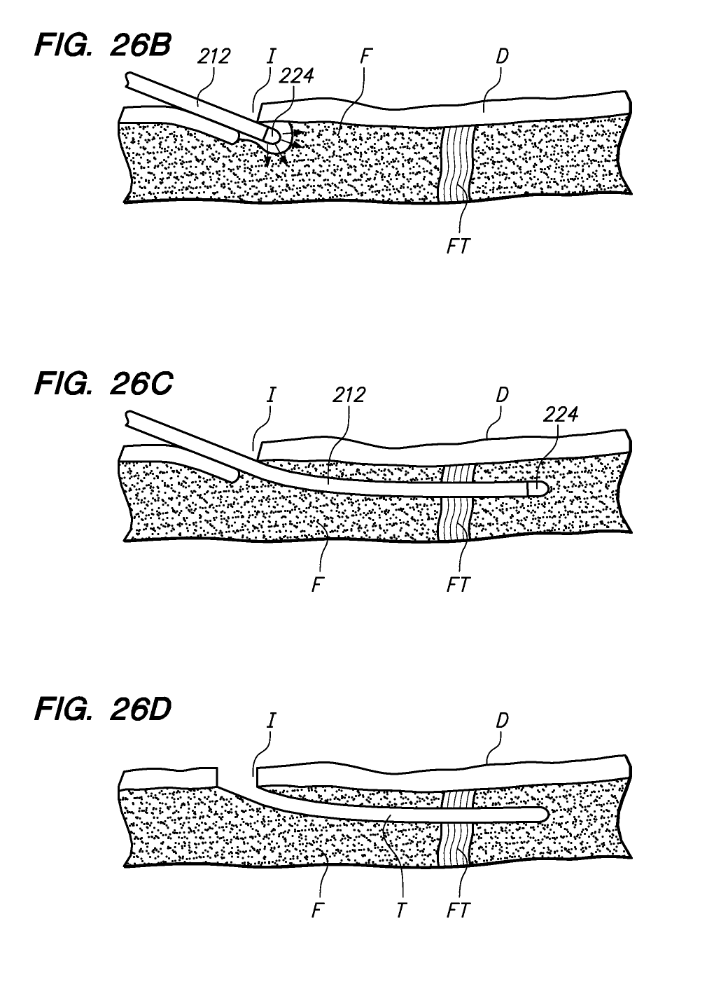

Referring to FIGS. 26A-26D, the operation of the tissue tunneling system 210 is described in subcutaneously creating a tunnel T within a patient. First, a small surgical incision I is made through the derma D of the patient using conventional means, and the atraumatic distal tip of the tunneling probe 212 is introduced into the incision I (FIG. 26A). Then the RF generator 214 is coupled to the connector 230 (shown in FIG. 24) of the tunneling probe 212 via RF cable 216, and while applying a moderate amount of axial pressure to the tunneling probe 212, RF energy is conveyed from the RF generator 214, through the shaft 218 (shown in FIG. 25), and out of the tip electrode 224 (shown by arrows), thereby ablating tissue immediately adjacent the tip electrode 224 (FIG. 26B). As a result, the distal tip of the tunneling probe 212 readily advances along a layer of fat F just beneath the derma D (FIG. 26C). Significantly, any fibrous tissue FT encountered by the tunneling probe 212 will readily be ablated, allowing the tunneling probe 212 to be advanced under the derma D without using brute force or cutting instruments.

If the tunneling probe 212 carries a sensor, a physiological parameter, such as impedance, temperature, or pressure, can be monitored to ensure that the distal tip of the tunneling probe 212 does not veer from the desired subcutaneous path into an unintended tissue structure, such as through a chest wall. For example, if the tip electrode 224 is used as an impedance sensor, the impedance between it and the indifferent patch electrode may be measured to detect an impedance change if the tip electrode 224 enters the unintended tissue structure. That is, if the tip electrode 224 enters another tissue structure from the layer of fat F, the measured impedance will conspicuously increase, thereby providing an indication that the tunneling probe 212 has veered away from the subcutaneous path, and that further advancement of the tunneling probe 212 should cease. Next, the tunneling probe 212 is removed from the patient, thereby leaving a tunnel T through which the conduit may be located (FIG. 26D). Notably, the surface of the tunnel T may be coagulated by the ablation energy, thereby minimizing or preventing blood loss within the tunnel T.

Although particular embodiments of the present inventions have been shown and described, it should be understood that the above discussion is not intended to limit the present inventions to these embodiments. It will be obvious to those skilled in the art that various changes and modifications may be made without departing from the scope of the appended claims.

1. A method of percutaneously accessing a patient with an elongated probe, comprising:
placing an atraumatic distal tip of the probe against the derma of the patient;
conveying electrical energy to or from the distal tip to ablate tissue immediately adjacent the distal tip; and
advancing the probe through the derma while the tissue immediately adjacent the distal tip is ablated.

2. The method of claim 1, wherein the electrical energy is only conveyed to or from the distal tip.

3. The method of claim 1, further comprising introducing a medical agent into the patient using the probe.

4. The method of claim 1, wherein the electrical energy is radio frequency (RF) energy having a power level equal to or less than 30 W.

5. The method of claim 1, wherein tissue immediately axial to the distal tip is ablated.

6. The method of claim 1, further comprising introducing a cannula over the probe.

7. The method of claim 1, further comprising introducing a guidewire through the probe.

8. The method of claim 7, wherein the electrical energy is conveyed between the distal tip and a catheter having the guidewire disposed therethrough, the method further comprising advancing the catheter with the guidewire through the derma while the tissue immediately adjacent the distal tip is ablated.

9. The method of claim 1, wherein the probe includes an outer cannula and an inner member, the method further comprising:
removing the inner member from the cannula; and
introducing a medical probe through the cannula into the patient.

10. The method of claim 1, further comprising introducing fluid into the patient or removing fluid from the patient through the probe.

11. The method of claim 1, further comprising intravascularly advancing the distal tip into the patient.

12. The method of claim 1, further comprising:
sensing physiological information adjacent the distal tip of the probe; and
determining the nature of tissue in which the distal tip is located based on the sensed physiological information.

13. A method of subcutaneously creating a tunnel through tissue with an elongated tunneling probe, comprising:
conveying electrical energy to or from an atraumatic distal tip of the tunneling probe to ablate tissue immediately adjacent the distal tip; and
subcutaneously advancing the tunneling probe within the patient while the tissue immediately adjacent the distal tip is ablated.

14. The method of claim 13, wherein the electrical energy is only conveyed to or from the distal tip.

15. The method of claim 13, wherein the electrical energy comprises radio frequency (RF) energy having a power level equal to 30 W or less.

16. The method of claim 13, further comprising:
sensing physiological information adjacent the distal tip of the tunneling probe; and
determining the nature of tissue in which the distal tip is located based on the sensed physiological information.

17. A medical probe, comprising:
an elongated, rigid, electrically conductive, shaft; and
an electrically insulative sheath disposed on the shaft to form an exposed atraumatic tip electrode configured for electrosurgically ablating solid tissue located immediately axial to the tip electrode to facilitate rapid advancement of the medical probe through the solid tissue without substantially ablating solid tissue immediately radial to the tip electrode.

18. The medical probe of claim 17, wherein the exposed tip electrode is confined to a distal-facing surface of the shaft.

19. The medical probe of claim 17, wherein the combination of the shaft and sheath has a size between 27 gauge and 8 Fr, and wherein the tip electrode is configured for ablating the solid tissue distal to the tip electrode when electrical energy at a power level of 30 W or less is applied to a proximal end of the shaft.

20. The medical probe of claim 17, further comprising a sensor carried by the shaft adjacent the tip electrode, the sensor configured for sensing a physiological parameter indicative of a tissue characteristic.

21. The medical probe of claim 17, wherein the probe defines a lumen for introducing a guidewire.

22. A percutaneous access system, comprising:
a catheter having a lumen; and
a guidewire configured for being introduced through the catheter lumen, wherein the guidewire includes an electrically conductive, shaft and an electrically insulative sheath disposed on the shaft to form an exposed atraumatic tip electrode configured for electrosurgically ablating solid tissue located immediately axial to the tip electrode to facilitate rapid advancement of the guidewire through the solid tissue without substantially ablating solid tissue immediately radial to the tip electrode.

23. The system of claim 22, wherein the exposed tip electrode is confined to a distal-facing surface of the guidewire shaft.

24. The medical probe of claim 22, wherein the tip electrode is configured for ablating the solid tissue distal to the tip electrode when electrical energy at a power level of 10 W or less is applied to a proximal end of the guidewire shaft, and wherein the combination of the shaft and sheath has a size between 27 gauge and 8 Fr.

25. The medical probe of claim 24, further comprising an electrical connector electrically coupled to the shaft.

26. The medical probe of claim 22, further comprising a sensor carried by the shaft adjacent the tip electrode, the sensor configured for sensing a physiological parameter indicative of a tissue characteristic.

27. A catheter assembly, comprising:
a flexible therapeutic or diagnostic catheter having an elongated shaft and a lumen axially extending through the catheter shaft; and
a flexible guidewire configured for being removably introduced through the catheter lumen, the guidewire having an elongated, electrically conductive, shaft and an electrically insulative sheath disposed on the guidewire shaft to form an exposed atraumatic tip electrode that extends from a distal end of the catheter shaft when the guidewire is inserted within the catheter lumen, the tip electrode configured for electrosurgically ablating solid tissue located immediately axial to the tip electrode to facilitate rapid advancement of the catheter through solid tissue.

28. The catheter assembly of claim 27, wherein the tip electrode is configured for electrosurgically ablating solid tissue located immediately axial to the tip electrode without substantially ablating solid tissue immediately radial to the tip electrode.

29. The catheter assembly of claim 27, wherein the tip electrode is confined to a distal-facing surface of the guidewire shaft.

30. The catheter assembly of claim 27, wherein the tip electrode is configured for ablating the solid tissue distal to the tip electrode when electrical energy at a power level of 30 W or less is applied to a proximal end of the guidewire shaft.

31. The catheter assembly of claim 27, further comprising an electrical connector electrically coupled to the guidewire shaft.

32. The catheter assembly of claim 31, further comprising a handle carrying the electrical connector and removably mounted to the guidewire shaft.

33. The catheter assembly of claim 27, further comprising a locking mechanism configured for alternately affixing the guidewire relative to the catheter and allowing the guidewire to move relative to the catheter.

34. The catheter assembly of claim 27, further comprising a sensor carried by the guidewire shaft adjacent the tip electrode, the sensor configured for sensing a physiological parameter indicative of a tissue characteristic.

35. A medical probe, comprising:
an elongated, electrically conductive, shaft;
a lumen extending through the shaft; and
an electrically insulative sheath disposed on the shaft to form an exposed atraumatic tip electrode configured for electrosurgically ablating solid tissue located immediately axial to the tip electrode to facilitate rapid advancement of the medical probe through the solid tissue.

36. The medical probe of claim 35, wherein the exposed tip electrode is confined to a distal-facing surface of the shaft.

37. The medical probe of claim 35, further comprising an electrical connector electrically coupled to the shaft.

38. The medical probe of claim 35, further comprising a sensor carried by the shaft adjacent the tip electrode, the sensor configured for sensing a physiological parameter indicative of a tissue characteristic.

39. The medical probe of claim 35, wherein the shaft is rigid.

40. A method of percutaneously introducing a guidewire into a patient, comprising:
placing an atraumatic distal tip of the guidewire against the derma of the patient;
conveying electrical energy to or from the distal tip to ablate tissue immediately adjacent the distal tip; and
advancing the catheter with the guidewire through the derma while the tissue immediately adjacent the distal tip is ablated.

41. The method of claim 40, wherein the electrical energy is only conveyed to or from the distal tip.

42. The method of claim 40, further comprising advancing the catheter with the guidewire into a blood vessel of the patient.

43. The method of claim 40, wherein the electrical energy is radio frequency (RF) energy having a power level equal to or less than 30 W.

44. The method of claim 40, wherein tissue immediately axial to the distal tip is ablated to a depth of no greater than 1 mm from the surface of the distal tip.

45. The method of claim 40, further comprising:
sensing physiological information adjacent the distal tip of the guidewire; and
determining the nature of tissue in which the distal tip is located based on the sensed physiological information.

46. A catheter assembly, comprising:
a flexible therapeutic or diagnostic catheter having an elongated shaft and a lumen axially extending through the catheter shaft;
a flexible guidewire configured for being removably introduced through the catheter lumen, the guidewire having an elongated, electrically conductive, shaft and an electrically insulative sheath disposed on the guidewire shaft to form an exposed atraumatic tip electrode that extends from a distal end of the catheter shaft when the guidewire is inserted within the catheter lumen; and
an electrical connector electrically coupled to the guidewire shaft,
wherein the tip electrode is configured for electrosurgically ablating solid tissue located immediately axial to the tip electrode without substantially ablating solid tissue immediately radial to the tip electrode.
